(19) United States
(12) Patent Application Publication
(10) Pub. No.: US 2026/0108210 A1
(43) Pub. Date: Apr. 23, 2026

(54) ELECTRONIC ACCESSORIES FOR WEARABLE DEVICES

(71) Applicant: Kandu Health, Inc., Campbell, CA (US)

(72) Inventors: Lindsay Howard, San Jose, CA (US); Kirsten Carroll, San Francisco, CA (US); Stefan Heuser, Piedmont, CA (US)

(21) Appl. No.: 19/477,459

(22) PCT Filed: Apr. 24, 2024
(86) PCT No.: PCT/US2024/025953

§ 371 Date: Oct. 21, 2025

Related U.S. Application Data
(60) Provisional application No. 63/462,948, filed on Apr. 28, 2023.

(51) Int. Cl. A61B 5/00; A61B 5/0205; A61B 5/026; A61F 7/00

(52) U.S. Cl. CPC A61B 5/681; A61B 5/02055; A61F 7/007; A61B 5/0261; A61F 2007/0035; A61F 2007/0093

(57) ABSTRACT

A system is described that includes an accessory device configured to removably attach to a wearable physiological monitoring device including at least an optical sensor. The accessory device can include a heat stimulus source and a battery configured to power the heat stimulus source. The accessory device can also include a housing having a first opening configured to enable the optical sensor of the wearable physiological monitoring device to contact a skin of a user wearing the wearable physiological monitoring device and/or a second opening configured to enable a display of the physiological monitoring device to be viewable through the second opening. The heat stimulus source can be positioned along at least some portions of a periphery of the first opening. The heat stimulus source can include concentric heating elements. The wearable physiological monitoring device can be configured to be secured to a wrist of a user.

### RELATED APPLICATIONS

This application claims the priority benefit of U.S. Provisional Patent Application 63/462,948 filed on Apr. 28, 2023, entitled “ELECTRONIC ACCESSORIES FOR WEARABLE DEVICES,” which is incorporated by reference herein in its entirety.

### BACKGROUND

### Field

This disclosure relates generally to the field of wearable devices for physiological signal monitoring, and in particular, to accessories for wearable devices.

### Description of the Related Art

In recent years, various wearable devices have been provided to users to allow users to access various content and information. Such wearable devices may be worn on the body of a user. Although these wearable device can improve portability of data and information, such devices may be limited with respect to assessing and providing information from the user and/or the environment around the user.

### SUMMARY

For purposes of summarizing the disclosure and the advantages achieved over the prior art, certain objects and advantages of the disclosure are described herein. Not all such objects or advantages may be achieved in any particular embodiment. Thus, for example, those skilled in the art will recognize that the invention may be embodied or carried out in a manner that achieves or optimizes one advantage or group of advantages as taught herein without necessarily achieving other objects or advantages as may be taught or suggested herein.

An improved way of adding or upgrading functionality of an electronic wearable device is described. In some implementation, an accessory system configured for use with a wearable electronic device can include: an electronic band configured to be removably coupled to a wearable electronic device, wherein the electronic band defines a first opening and a second opening and includes: an external surface opposite an internal surface, and an electronics module provided on the external surface of the electronic band, the electronics module including: a sensor system configured to be communicatively coupled to the wearable electronic device when the wearable electronic device is connected to the electronic band, the sensor system being at least partially in contact with a portion of the internal surface of the electronic band through the first opening in the electronic band, and a stimulus source including a first surface area extending through the second opening of the electronic band to be positioned within the internal surface of the electronic band.

In some implementations, the accessory system further includes a first coupling part provided on a first end of the electronic band and a second coupling part provided on a second end of the electronic band, wherein one or more of the first coupling part and the second coupling part are configured to removably couple the electronic band to the wearable electronic device to cause a communicative connection between the sensor system and the wearable electronic device. In some implementations, the first coupling part or the second coupling part is configured to electrically couple to a connection terminal of the wearable electronic device.

In some implementations, the first coupling part includes a first connector and a second connector, the first connector being inline with the second connector, the second coupling part includes a third connector and a fourth connector, the third connector being inline with the fourth connector, and any one of the first connector, the second connector, the third connector, and the fourth connector are configured with connector circuitry for connecting to at least one connection terminal of the wearable electronic device to cause the electronic band to be in electrical communication with the wearable electronic device when the respective connector is coupled to the at least one connection terminal of the wearable electronic device.

In some implementations, the sensor system includes a plurality of sensors, the plurality of sensors including one or more of: a pressure sensor, a motion sensor, a skin temperature sensor, a blood volume sensor, a muscle activity sensor, a sweat sensor, a tissue impedance sensor, or an environmental temperature sensor. In some implementations, the electronic band is one or more of: adjustable, stretchable, or tensionable. In some implementations, the wearable electronic device is at least one of: a sensor patch, a watch, a smart watch, a fitness tracker, a phone, a wearable battery, a camera, a bracelet, a ring, a controller, and smart glasses.

In some implementations, an accessory interface can include: a communication means for transmitting and receiving data between an electronic accessory and a wearable device, wherein the electronic accessory includes a first sensor system and the wearable device includes a second sensor system; detection circuitry to selectively couple the first sensor system to the second sensor system, the detection circuitry being configured to: receive a request for sensing at least one physiological parameter of a user wearing the electronic accessory and the wearable device; and in response to determining that the second sensor system includes at least one sensor operable to generate additional measurements corresponding to the at least one physiological parameter, selectively couple control of the second sensor system to the electronic accessory to carry out operations including: synchronizing the first sensor system with the second sensor system, detecting a first output signal associated with the first sensor system, transmitting a plurality of control signals to the second sensor system based on the first output signal, receiving a second output signal from the second sensor system, the second output being based on the plurality of control signals, and generating the at least one physiological parameter based on the first output signal from the first sensor system and the second output signal from second sensor system.

In some implementations, a system for retaining a wearable electronic device can include: a flexible housing defining an open end opposite a base, wherein the open end is further defined by a perimeter effective for retaining a first portion of the wearable electronic device, and the base is configured to retain a second portion of the wearable electronic device, wherein the flexible housing further includes: a stimulus source including a surface area extending on a first portion of a surface of the base and through a first opening defined by the surface of the base; and at least one sensor positioned on a second portion of the surface of the base and in a second opening defined by the surface of the base, the stimulus source being in electrical contact with the at least one sensor.

In some implementations, the base includes at least one connection port configured to electrically connect the at least one sensor and the stimulus source to a receiving port of the wearable electronic device when the wearable electronic device is retained in the flexible housing. In some implementations, the system further includes: at least one processor communicatively coupled to the at least one sensor and the stimulus source; a transceiver communicatively connectable to the at least one processor, the stimulus source, the at least one sensor, and the wearable electronic device, wherein the at least one processor is configured to execute computer-readable instructions including: monitoring a body site of a person having at least partial contact with the at least one sensor; obtaining, based on the monitoring, a physiological signal associated with the body site; and generating, based on the physiological signal, a vasodilation estimate for the body site, wherein the body site is placed in contact with the stimulus source of the wearable electronic device.

In some implementations, obtaining the physiological signal occurs after completion of at least one stimulus cycle generated by the stimulus source. In some implementations, the system further includes at least one input device configured to control a plurality of functions of the flexible housing and one or more functions of the wearable electronic device. In some implementations, the wearable electronic device is at least one of a sensor patch, a watch, a smart watch, a fitness tracker, a phone, a wearable battery, a camera, a bracelet, a ring, a controller, and smart glasses.

In some implementations, an accessory device configured to removably attach to a wearable physiological monitoring device including at least an optical sensor can include: a heat stimulus source; and a battery configured to power the heat stimulus source.

In some implementations, the accessory device further includes a housing, said housing including a first opening, said first opening configured to enable the optical sensor of the wearable physiological monitoring device to contact a skin of a user wearing the wearable physiological monitoring device. In some implementations, the housing further includes a second opening, said second opening being configured to enable a display of the physiological monitoring device to be viewable through the second opening.

In some implementations, the heat stimulus source is positioned along at least some portions of a periphery of the first opening. In some implementations, the heat stimulus source includes concentric heating elements.

In some implementations, the housing includes side walls and wherein the battery is positioned along at least one of the side walls. In some implementations, the accessory device further includes a hardware processor configured to control the heat stimulus source. In some implementations, the accessory device further includes one or more antennas configured to communicate with the wearable physiological monitoring device.

In some implementations, the accessory device further includes one or more antennas configured to communicate with a computing device. In some implementations, the physiological monitoring device does not include any optical sensor. In some implementations, the accessory device further includes one or more temperature sensors.

In some implementations, the wearable physiological monitoring device is configured to be secured to a wrist of a user. In some implementations, the accessory device further includes a strap configured to secure the wearable physiological monitoring device to the user, wherein at least some components of the accessory device are integrated into the strap.

In some implementations, the heat stimulus source is positioned outside of an outer periphery of the wearable physiological monitoring device. In some implementations, the accessory device further includes a strap configured to removably attach to the wearable physiological monitoring device, wherein the accessory device is integrated into the strap.

In some implementations, a strap configured to secure a wearable physiological monitoring device to a wrist of a user, said wearable physiological monitoring device including at least an optical sensor, can include: a heat stimulus source; and a battery configured to power the heat stimulus source.

In some implementations, the battery is positioned on an outer surface of the strap not in contact with a skin of the user. In some implementations, the strap further includes an optical sensor enclosed within an area of the heat stimulus source.

In some implementations, the optical sensor is positioned on the strap to measure a vasodilation response on a ventral side of the wrist. In some implementations, the heat stimulus source substantially covers an inner surface of the strap that is in contact with a skin of the user.

In some implementations, the heat stimulus source include a plurality of heat stimulus sources, wherein a first heat stimulus source is positioned adjacent to a first connector and wherein a second heat stimulus source is positioned adjacent to a second connector. In some implementations, the heat stimulus source is integrated within an outer surface and an inner surface of the strap, thereby hidden within the strap.

In some implementations, a wearable receptacle structure can include: a receptacle portion including a perimeter and an opening, said opening configured to receive a physiological monitoring device, wherein said perimeter includes a first heat stimulus source; and a strap configured to secure the receptacle portion to a body of a user.

In some implementations, the receptacle portion and the strap form a unitary body. In some implementations, a portion of the perimeter of the receptacle structure further includes a power source configured to provide power to the first heat stimulus source.

In some implementations, the strap includes a second heat stimulus source. In some implementations, the perimeter includes one or more walls, said one more walls including a volume configured to house electronics and a power source. In some implementations, the physiological monitoring device is detachable from the receptacle portion.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various implementations, with reference made to the accompanying drawings.

FIG. 1A illustrates an example of a wearable device for monitoring physiological parameters of a user.

FIG. 1B illustrates a block diagram of an example physiological monitoring system for obtaining and processing biological data.

FIG. 1C illustrates a block diagram of an example computing environment for processing biological data.

FIG. 2 illustrates a flow diagram of an example process for generating biological data characterizations and/or estimates.

FIG. 3 illustrates a first and second wearable device for measuring biological data and response asymmetry across a right and left limb, respectively.

FIG. 4A illustrates a perspective view of an example implementation of a wearable device for obtaining biological data.

FIG. 4B illustrates a perspective view of the wearable device of FIG. 4A.

FIG. 4C illustrates a partial view of the wearable device of FIG. 4A.

FIG. 4D illustrates a rotated view of the wearable device of FIG. 4C.

FIG. 4E illustrates a perspective view and partial exploded view of the wearable device of FIG. 4C.

FIG. 4F illustrates a front view of the wearable device of FIG. 4C.

FIG. 5A illustrates a perspective view of an example implementation of an electronic auxiliary case for obtaining biological data.

FIG. 5B illustrates a side perspective view of the electronic auxiliary case of FIG. 5A.

FIG. 5C illustrates a bottom view of the electronic auxiliary case of FIG. 5A.

FIG. 5D illustrates a perspective view of an example implementation of another electronic auxiliary case for obtaining biological data.

FIG. 6A illustrates a perspective view of an example implementation of an electronic band for obtaining biological data.

FIG. 6B illustrates a perspective view of another example implementation of an electronic band for obtaining biological data.

FIG. 7 illustrates a perspective view of an example implementation of an electronic auxiliary case for obtaining biological data.

FIG. 8 illustrates a top down view of yet another example implementation of an electronic band for obtaining biological data.

FIG. 9 is an example physiological monitoring system for obtaining and processing biological data.

FIG. 10 illustrates a flow diagram of an example process for characterizing or monitoring a physiological response of a person using an auxiliary device.

FIG. 11A illustrates a perspective view of an example of an accessory device coupled to a wearable physiological monitoring device.

FIG. 11B illustrates a perspective view of the accessory device of FIG. 11A.

FIG. 11C illustrates another perspective view of the accessory device of FIG. 11A.

FIG. 12 illustrates a perspective view of another example accessory device.

The illustrated implementations are merely examples and are not intended to limit the disclosure. The schematics are drawn to illustrate features and concepts and are not necessarily drawn to scale.

### DETAILED DESCRIPTION

Disclosed herein are systems and methods for obtaining, monitoring and/or characterizing a vasodilation response of a user. For example, the systems may include wearable devices that can stimulate a skin surface site to obtain the vasodilation response of a user wearing such devices. Appendix A (incorporated herein in its entirety) of PCT Application PCT/US2022/071701 filed on Apr. 28, 2022, shows example wearable systems and methods for measuring vasodilatory response.

Described herein are systems, devices, and methods for electronics and electronic accessories for adding functionality to a wearable device (e.g., a wearable electronic device 102, 530, or the like). The electronic accessories described herein may include auxiliary electronics and/or sensors that may monitor, measure, or otherwise obtain physiological parameters of a user. These auxiliary electronics may add functionality or improve upon functionality of a wearable device and/or may share monitoring tasks and data tasks with sensors, data, and other hardware on the wearable electronic device to provide added functionality for the user.

The electronic accessories described herein may be physically and/or communicatively coupled to a wearable device such that the electronic accessories add main or auxiliary functionality/monitoring to the wearable device. In some implementations, an electronic accessory may be in the form of a case or protective case for the wearable device. In some implementations, an electronic accessory may be in the form of a band for the wearable device. In some implementations, the electronic accessory may function with a wearable device wirelessly, such that the accessory and the wearable device can communicate over a shared network, for example, using, a nearfield communications (NFC) protocol, a low energy Bluetooth® protocol, other radiofrequency (RF) communication protocol, and/or Wi-Fi, etc. In some implementations, the electronic accessory may function with a wearable device through a wired or electrical connection between the electronic accessory and the wearable device.

### Wearable Physiological Monitoring Device

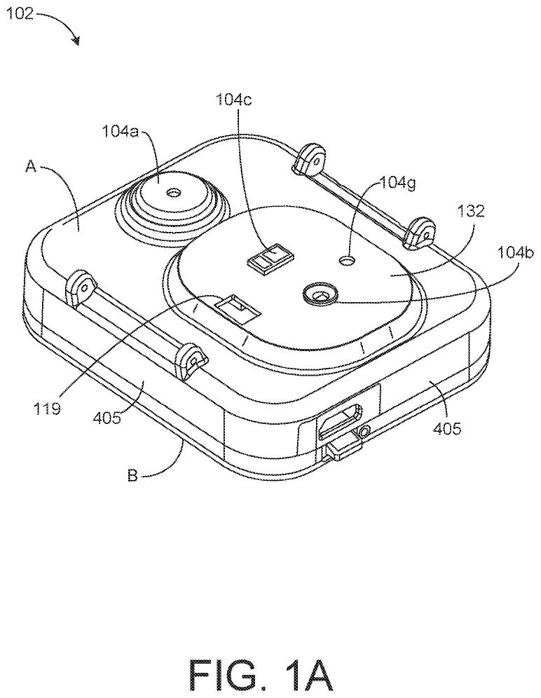

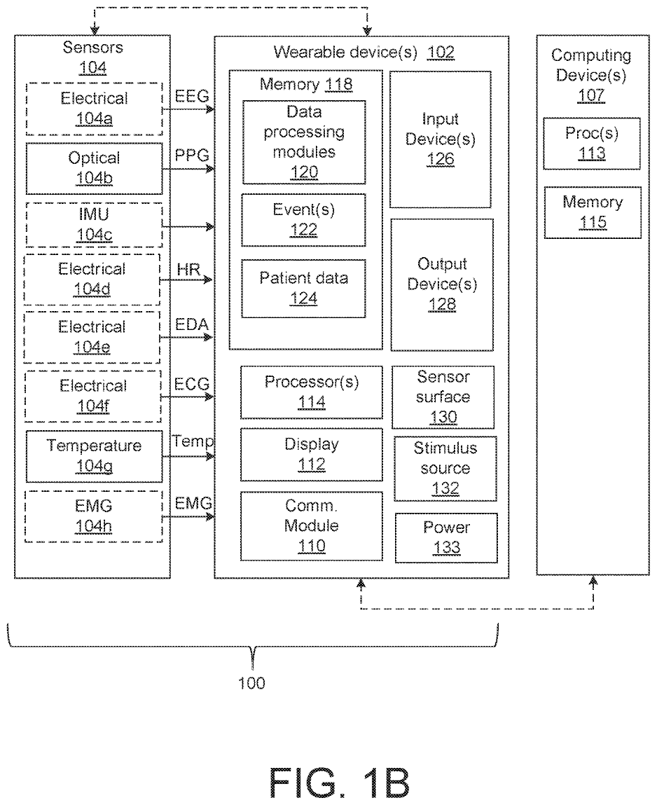

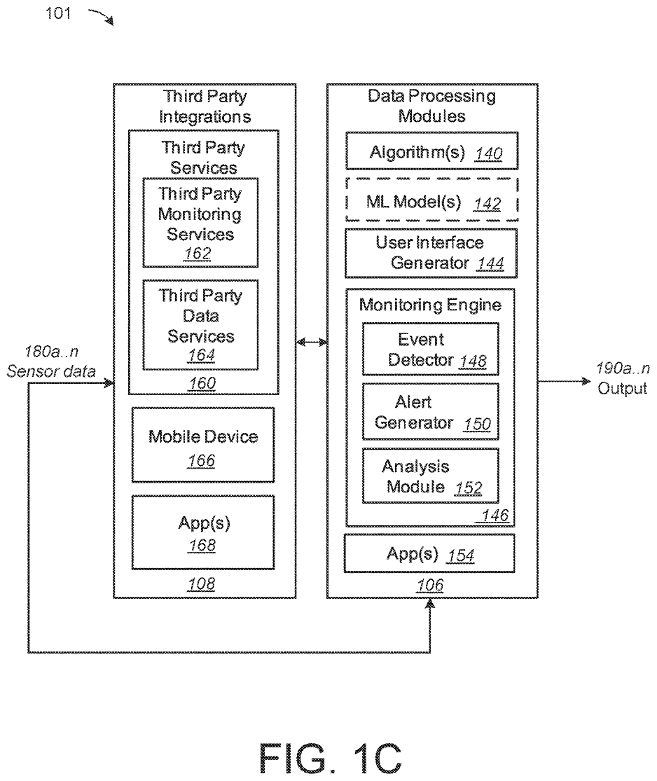

FIG. 1A illustrates an example of a wearable device 102 for monitoring physiological parameters of a user (e.g., a person, a patient). The wearable device 102 can include one or more sensors for detecting physiological parameters of the user. For example, the wearable device 102 can include a sensor 104a (e.g., an electrical sensor) for obtaining temperature responses and/or EDA responses from a skin surface site of the user. The wearable device 102 can also include an optical sensor 104b for obtaining a temperature and/or a photoplethysmographic (PPG) measurement from a skin surface site of the user. Other sensors and/or elements can be incorporated into or near to the device 102, as will be described in detail below.

The wearable device 102 can be configured to measure any number of physiological parameters of a user wearing the device 102. The measurements can be retrieved using one or more sensors integrated into or associated with device 102. For example, the device 102 can include one or more sensors for measuring core and/or skin surface temperatures; volumetric impedance spectroscopy; hyperhidrosis; heart rate or heart rate variability; and/or motion (e.g., by including an accelerometer and/or gyroscope therein) to measure, for example, limb asymmetry or changes in gait. In particular, device 102 can include one or more of a pressure sensor, a motion sensor, a skin temperature sensor, a blood volume sensor, a muscle activity sensor, a sweat sensor, a tissue impedance sensor, or an environmental temperature sensor. In addition, the device 102 can be communicatively coupled (e.g., via antenna, coils, etc.) to an external sensor that is not housed in or integrated into device 102.

Some or all components of the wearable device 102 can be integrated into a patch, a band, a watch, an adhesive strip, a case, a bracelet, an anklet, a sock, a shoe insole, a shoe, clothing, or any other wearable accessory. For example, some or all components of the wearable device 102 can be incorporated into a ring or band or a pair of rings or bands to be worn one on each arm, wrist, finger, leg, ankle, or foot. In some implementations, the ring or band can incorporate a stretchable or expandable element or stretch sensor to allow the ring or band to expand or stretch when the limb or limb portion, etc. swells. This element can include, but is not limited to, elastomer film polymers of various degree of bonding to allow for different pliable elements or measuring the reflectivity of polarized light. This element can include a plastic segment of the ring or band that can be loosened/tightened, or by building a slidable element that can be pulled apart. Non-limiting examples of a stretch sensor include, but are not limited to, a strain gauge or an electrical component which can change inductance, resistance, or capacitance when stretched.

Referring again to FIG. 1A, the wearable device 102 includes a body portion with a first surface A opposite a second surface B. The first surface A can be placed in contact with a skin surface of a user to obtain measurements and provide a stimulus via a stimulus source 132, for example heat or coolness via a thermal stimulus source. The first surface A and second surface B can be coupled via one or more or a plurality of sidewalls of device 102. For example, one or more sidewalls 405 can extend from a perimeter of the first surface A and couple to a perimeter of the second surface B. The first surface A and/or second surface B can include one or more sensors 104 (e.g., sensor or electrode 104a, sensors 104b, 104c, 104d, 104e, 104f, 104g, and/or 104h, etc. as shown in FIGS. 1A-1B) positioned thereon. For example, one or more sensors 104 on the first surface A can measure an environment of the user wearing or using the wearable device 102, and one or more sensors on the second surface B can measure one or more properties, features, or characteristics of the skin surface of the user. In some implementations, the wearable device 102 can include an opening, such as opening 119, to allow the heat source 132 to communicate with and/or access other components inside the body of device 102, for example processor(s) 114 and/or power source 133.

The wearable device 102 can be worn on an exterior or skin surface of a user (e.g., a patient). In some implementations, the wearable device 102 can include a strip or form a strip that measures muscle contractions through surface electromyography (sEMG). The measurement of EMG can be compared to a baseline value to detect a change or asymmetry of the EMG. In some implementations, EMG measures a user's intent to move a muscle and a corresponding actual user movement of the same muscle, compared to a typical movement profile of the muscle or a movement response on an opposite limb or extremity.

In some implementations of device 102, one or more sensors/electrodes 104a can be integrated into a body of the wearable device 102 or in a separate component of the wearable device 102. Further, in some implementations of the device 102, a stimulus source 132 is not utilized when monitoring and/or characterizing a vasodilation response. In a non-limiting example of a wearable device without a stimulus source 132, fluid shift within the vasculature (e.g., when someone moves from laying to sitting to standing) can be sufficient to detect vasodilation as a result of the fluid shift. For example, the wearable device 102 can detect limb position or perform pulse wave analysis, to evaluate aspects of vasodilation. In one exemplary, non-limiting example, when the arm is up in the air, the blood drains out of the hand resulting in the photoplethysmogram alternating current (AC) amplitude dropping. With the arm down by the side, the AC amplitude can increase because of blood pooling in the hand. The same effects occur in the feet when moving between standing, sitting, and laying down due to gravity induced blood pooling. A communicatively coupled accelerometer or an accelerometer embedded in the wearable device 102 can be used to detect limb position so that limb position can be tracked and a blood volume signal recorded based on the detected position.

In some implementations, the wearable device 102 can generate a stimulus for delivery to a monitoring site and measure a response in physiological parameters based on the stimulus. The stimulus can be applied to one location or a plurality of locations. In one implementation, the stimulus is applied bilaterally (e.g., to detect asymmetrical responses) on the body of the user to determine whether the response or the difference in response between the two sides indicates an atypical event such as a stroke event, or otherwise a deviation from baseline. For example, the stimulus can be applied in a stimulus cycle such that the baseline, during stimulation, and post stimulation responses are measured, or change in (e.g., slope, decay, etc.) responses between different measurement periods are determined. For example, a thermal (i.e., hot or cold) stimulus can be applied to a section of skin on a body of a user (shown in top panel) and the body's response to the stimulus source can be monitored over time (shown in bottom panel) to determine whether homeostasis is reached and/or a difference in response or return rate exists between the two sides of the body (in other words, determine whether an asymmetrical response exists). Responses can be indicative of changes in or perturbations in the parasympathetic nervous system, the sympathetic nervous system, the central vascular system, or the peripheral vascular system.

Further examples include stimulating the muscular or nervous system using electrical signals and monitoring the response over time and/or between sides using electromyogram (EMG), bioimpedance, or electroneurogram (ENG), respectively. These “stimulators/transmitters” and “receivers/detectors” could be in the same region or could be separated to measure across regions of the body.

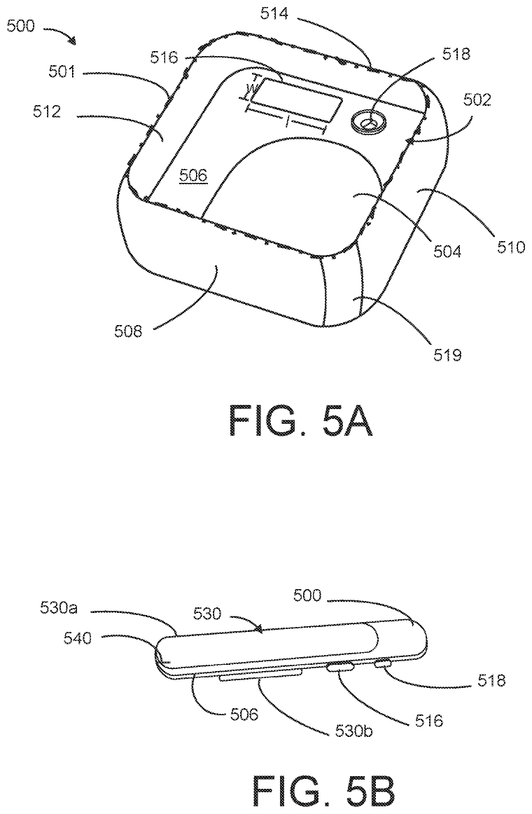

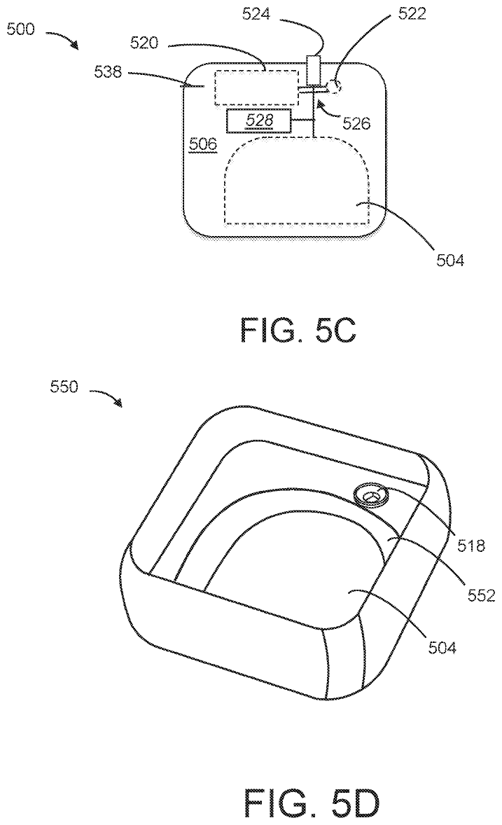

The wearable device 102 can be worn on an exterior or skin surface of a user (e.g., a patient) prior to, during, and/or after an anomalous biologic event, including up to days before the event, during the event, and/or after the event to provide continuous variable monitoring of various physiological parameters. For example, the wearable device 102 can function to monitor or characterize a vasodilation response in response to application of a stimulus (heating or cooling) or as a result of fluctuations in vasodilation (without a stimulus source). In some implementations, the wearable device 102 can be encased in an electronic case that can supplement or add to the functionality of the wearable device 102, by adding any or all of a stimulus source, additional sensors, additional processing devices, additional power devices or electronics, additional notification/communication devices (e.g., user interfaces, indicator lights, speakers, haptic components, microphones, etc.), cameras, and/or devices that function to combine functionality amongst sensors on device 102 and sensors on the electronic accessory, as will be described in detail in at least FIGS. 5A-9.

In some implementations, the device 102 can include and/or communicate with a device positionable in a room, office, home, vehicle, or other location; or in or on a bed or other furniture (e.g., bedside monitors; monitors within mattresses, bedding, etc.). For example, a smart speaker (e.g., to prompt a user to respond to a question to analyze speech quality), microphone, camera, and/or mirror can be positionable in a location to detect changes in a user's speech, activities, movement, gait, facial appearance, heart rate, and/or heart rate variability or changes from baseline. The device can include one or more data processing modules to differentiate changes in the measured parameters as compared to that from healthy learned patient data or individualized baseline data.

### Physiological Monitoring System

FIG. 1B illustrates a block diagram of a physiological monitoring system 100. The system 100 can include a device 102 as described above and one more sensors 104. The system 100 can measure, characterize, or detect physical responses (e.g., stroke events, stress response, heat stroke, seizure, menopause, diabetes, etc.) for a user wearing a wearable device that includes (or is in communication with) monitoring system 100. In some implementations, the sensors 104 can be integrated with the device 102. In some implementations, some or all of the sensors 104 can be physically separate from the device 102. The sensors 104 can be communicatively coupled to the wearable device 102 including wired and/or wireless connections. The system 100 can also connect with a computing device 107 as described below. Alternatively, or additionally, the system 100 can integrate with third-party devices and/or services (e.g., as shown in FIG. 1C).

In some implementations, the wearable device 102 can include a communication module 110, a display 112, processors 114, and memory 118. The display 112 cannot be included in all implementations. The communication module 110 can include one or more antennas or coils for wireless connections. The communication module 110 can also include components for wired connection, such as USB data transfer.

The processors 114 can include one or more hardware processors, including microcontrollers, digital signal processors, application specific integrated circuit (ASIC), a field programmable gate array (FPGA) or other programmable logic device, discrete gate or transistor logic, discrete hardware components, or any combination thereof designed to perform the functions described herein and/or capable of executing instructions, such as instructions stored by the memory 118. The processors 114 can also be able to execute instructions for performing communications amongst wearable device 102, sensors 104, data processing modules 106 (FIG. 1C), and/or third-party integrations 108 (FIG. 1C).

The memory 118 can include one or more non-transitory computer-readable storage media. The memory 118 can store instructions and data that are usable in combination with processors 114 to execute algorithms 140, optional machine learning models 142, monitoring engine 146 tasks, and apps 154. The memory 118 can also function to store or have access to the data processing modules 120, events 122, and patient data 124.

The system 100 can further include (or be communicatively coupled to) input devices 126, output devices 128, sensor interface 130, stimulus source 132, and/or power source 133 (e.g., battery). The input devices 126 can interact with one or more processors 114, memory 118, and/or sensors 104. The input devices 126 can include buttons, touchscreens, switches, toggles, and/or other hardware components located on wearable device 102. In some implementations, the input device 126, or at least some functionality of input device 126, can be external to or not integrated into the device 102, such that one or more controllers, mobile device (e.g., mobile device 166), apps (e.g., apps 168), etc., can communicate (e.g., antenna, coils, etc.) with device 102 using a wireless communications protocol. In some implementations, the input device 126 can include, for example, a touch input device that can receive tactile user input, a microphone that can receive audible input, and the like.

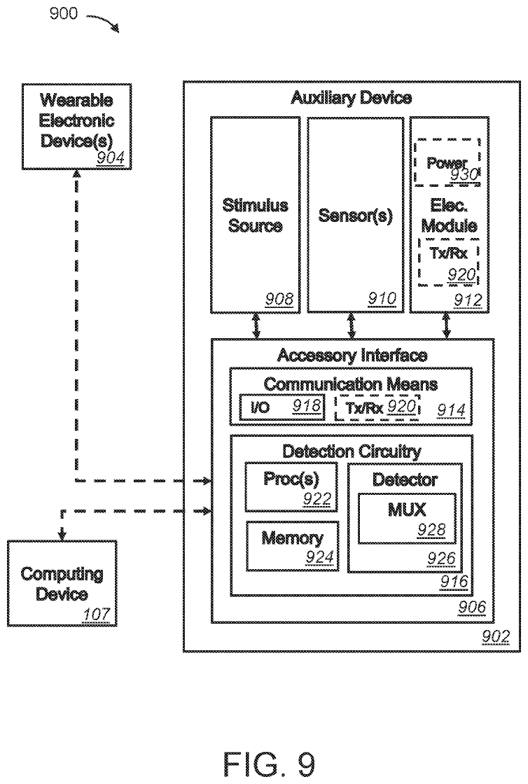

In some implementations, the input devices 126 (e.g., I/O 918 of FIG. 9) can include one or more sensor inputs to electrically and communicatively couple to one or more components (e.g., stimulus source 908 and/or sensors 910 (FIG. 9)) of an auxiliary device, such as a case, a band, a ring, or other device configured to couple to device 102. In some implementations, the input devices 126 can further couple sensors on device 102 to any or all accessories, stimuli, power sources, and/or user interfaces associated with the auxiliary devices described herein.

The output devices 128 (e.g., I/O 918 of FIG. 9) can interact with one or more processors 114, memory 118, and/or sensors 104. The output devices 128 can include, for example, a display (e.g., display 112) for visual output, a speaker for audio output, haptic feedback, or other interface for outputting data to another device. For example, the output devices 128 can include one or more sensor interfaces to electrically and communicatively couple to one or more components (e.g., stimulus source 908 and/or sensors 910 (FIG. 9)) of an auxiliary device, such as a case, a band, a ring, or other device configured to couple to device 102. In some implementations, the output devices 128 can further couple sensors on device 102 to any or all accessories, stimuli, power sources, and/or user interfaces associated with the auxiliary devices described herein.

The sensor interface 130 can store instructions to carry out operations pertaining to received sensor signals from one or more of the sensors 104. For example, the instructions can enable interaction with one or more processors 114, memory 118, and/or sensors 104 to communicate sensor data from one or more of the sensors 104 to the wearable device 102. The sensor data can be obtained from the sensors 104 taking recordings and measurements. The sensors 104 can include any or all the electrical sensors 104a, 104d, 104e, 104h, optical sensors 104b, 104g, and/or mechanical sensors (e.g., strain gauge, sensor 104c, etc.), as described in detail throughout this disclosure.

The stimulus source 132 can be a heating element, a thin film resistance flexible heater, a polyimide heater, an optical heater (e.g., a laser), and the like. In some implementations, the stimulus source 132 can be a cooling element, a thermoelectric cooler, a miniature compressor, or the like. The stimulus source 132 can be placed in communication with the skin surface. The stimulus source 132 can heat or cool the skin surface to a target offset temperature or a pre-determined temperature. In some implementations, the stimulus source 132 cannot be integrated into the wearable device and can instead be an environmental heat source, for example a warm room, warm enclosure, and/or other warm environment.

Temperature sensors 104g that can be integrated within and/or communicatively coupled to device 102 can include, but are not limited to, infrared sensors, thermometers, thermistors, or thermal flux transducers. Hyperhidrosis measurement devices can include, but are not limited to, detection of analytes including ions, metabolites, acids, hormones, and small proteins through potentiometry, chronoamperometry, cyclic voltammetry, square wave stripping voltammetry, or detection of changes in conductivity.

The power source 133 can include or connect to a battery or a port for connecting the device 102 to a power supply, a wall power adapter, or the like.

The sensors 104 can be controlled by one or more processors 114. Furthermore, the one or more processors 114 can also obtain and process sensor signal data. Additionally, the one or more processors 114 can communicate raw or processed sensor signal data to third-party integrations 108. The sensors 104 can include one or more of an electroencephalogram (EEG) sensor 104a, a blood volume sensor 104b (e.g., a photoplethysmographic (PPG) sensor), an inertial measurement unit (IMU) sensor 104c, a heart rate (HR) sensor 104d, an electrodermal activity (EDA) sensor 104e, an electrocardiogram (ECG) sensor 104f, a temperature sensor 104g (e.g., a skin temperature sensor), and an electromyography (EMG) sensor 104h and/or mechanical sensors (not shown).

Although various sensor technologies are described elsewhere herein, additional, non-limiting sensors that can be integrated into device 102 or communicatively coupled (e.g., antennas, coils, etc.) to device 102 include: a photoplethysmographic (PPG) device, a skin conductance sensor measuring skin conductance/galvanic skin response (GSR) or electrodermal activity (EDA), or a skin temperature measurement device (e.g., contact devices and non-contact devices, like IR imaging camera).

The EEG sensor 104a can include one or more electrodes that can detect abnormalities in electrical activity of the brain.

The blood volume sensor 104b can include an optical sensor to detect blood volume changes in tissue. The blood volume sensor 104b can measure volumetric impedance spectroscopy, heart rate, respiration rate, or heart rate variability through monitoring a rate of blood flow.

The IMU sensor 104c can monitor motion (e.g., by including an accelerometer and/or gyroscope therein) to measure, for example, limb asymmetry or changes in gait or limb movement.

The heart rate sensor 104d can include one or more electrical sensors (e.g., electrocardiogramsors (e.g., PPG), or mechanical sensors (e.g. accelerometer, strain gauge, pressure sensors) to measure and monitor a heart rate.

The EDA sensor 104e can measure hyperhidrosis. Hyperhidrosis measurement devices can include, but are not limited to, evaluating sweat rate; detection of analytes including ions, metabolites, acids, hormones, and small proteins through potentiometry, chronoamperometry, or cyclic voltammetry; or detection of changes in conductivity.

The ECG sensor 104f can include a plurality of electrodes for recording electrical signals of the heart, to determine heart rate, determine cardiac rhythm, cardiac autonomic control and other cardiovascular and cardiorespiratory metrics. In instances of a stroke event, changes in these signals can indicate a potential stroke state.

The temperature sensor 104g can measure temperature. Temperature sensors can include, but are not limited to, infrared sensors, infrared imaging cameras, thermometers, thermistors, or thermal flux transducers. Temperature sensors are used to measure the core body temperature and surface temperatures at various sites on the body. In a stroke event, there can be variations in temperature related to disruptions in temperature regulation, at the core and/or at individual surface sites, that can indicate a stroke event.

The EMG sensor 104h can include two or more or a plurality of electrodes for recording electrical signals of muscles. For example, an EMG sensor 104h can be used to measure an intent to move signal and a resulting movement signal of a muscle. In instances of a stroke event, the intent to move signal can be recorded but the movement signal can be absent, indicating a potential stroke state.

Additional metrics that can be obtained by device 102 with one or more additional sensors 104, processors 113 and/or processors 114, and/or electrodes can include, movements, reflexes, stimulus response output, breathing patterns, metrics responsive to audio tactile and/or audio input, etc.

In some implementations, the wearable device 102 can be communicatively coupled to a computing device 107. The computing device 107 can be a mobile device, a laptop device, a second wearable device, or the like. The computing device 107 can be a computer-executable component that includes processors 113 and memory 115 that can be used to execute the methods described herein. In some implementations, the computing device 107 can be used as a hub to send data to one or more server(s). The computing device 107 and/or particular software application(s) installed on the computing device 107 can also be used to control operation of the wearable device 102.

In some implementations, the system 100 or environment 101 (operating on device 102) can further include one or more software applications downloaded and/or stored on a hardware component of device 102 or on a hardware component of computing device 107. The application can process sensor data, electrode data, camera data, speech data, and/or display data sensed or captured in real time, for example in a graphical representation, of the data.

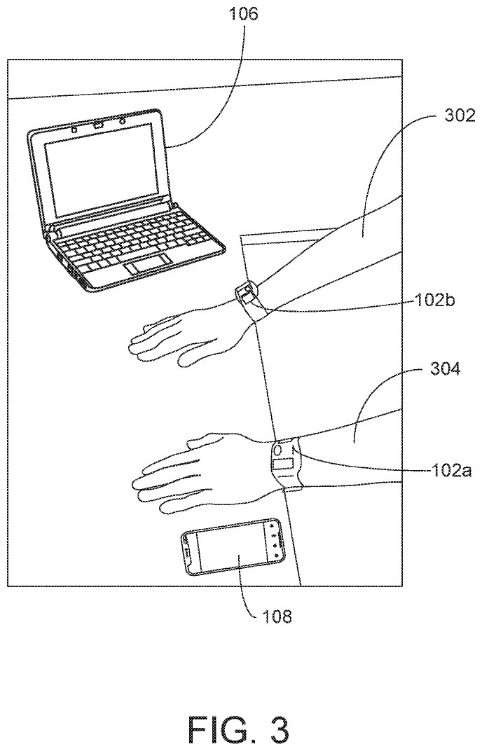

While the system 100 is depicted with a single wearable device 102, any number of wearable devices 102 can be included or communicatively coupled to system 100 (e.g., a first wearable device 102a and/or a second wearable device 102b of FIG. 3). In some implementations, when multiple wearable device(s) are employed, it is not necessary that all wearable devices include all of the components described above. For example, when two wearable devices 102 are used, one of the wearable devices can have limited processing power as all of the processing can be performed on a different wearable device 102. In some instances, one of the wearable devices can include a limited number of communication modules.

Data obtained with wearable device 102 can be transmitted to and/or from the system 100 (and/or environment 101 and/or computing device 107) to a central hub, mobile computing device (e.g., computing device 107), server, or other storage and/or computing device. Sharing of such data is performed according to user permissions. For example, a user of device 102 can configure data sharing permissions for device 102 to ensure that the data being shared is shared according to the configured user permissions.

Data transmissions described herein can include wireless communication (e.g., a nearfield communications (NFC) protocol, a low energy Bluetooth® protocol, other radiofrequency (RF) communication protocol, Wi-Fi, etc.) between sensor locations on the body and/or a central hub. In some implementations, data transmission can include wire communication between sensor locations on the body and/or a central hub. In some implementations, the central hub can be a monitor in a medical facility, home monitor, patients'mobile computing device, or other wireless device. Alternatively, one or more of the sensors on the body can act as the central hub. The hub device can wirelessly send signals to activate a medical care pathway and/or notify one or more individuals (e.g., family, friends, physician, EMS, etc.). In some implementations, data transmission, following multivariate analysis, to the central hub can alert the patient, the next of kin, and/or a third party to identify possible false positives or negatives.

### Processing System(s)

FIG. 1C illustrates a block diagram of an example computing environment 101 for processing biological data. Some or all aspects of the computing environment 101 can be implemented by the systems and devices described herein. As shown, the computing environment 101 includes data processing modules 106 and optional (shown by dashed lines) third-party integrations 108. The data processing modules 106 can include multiple engines for performing the processes and functions described herein. The engines can include programmed instructions for performing processes as discussed herein for detection of input conditions and control of output conditions. The engines can be executed by the one or more hardware processors of the device 102 alone or in combination with other hardware devices, such as the computing device 107. The programming instructions can be stored in a memory as discussed above. The programming instructions can be implemented in C, C++, JAVA, or any other suitable programming languages. In some implementations, some or all of the portions of the data processing modules 106 including the engines can be implemented in application specific circuitry such as ASICs and FPGAs. Some aspects of the functionality of the controller associated with data processing modules 106 can be executed remotely on a server (not shown) over a network. While shown as separate engines, the functionality of the engines as discussed below cannot be separate. Accordingly, the data processing modules 106 can be implemented with the hardware components described above with respect to FIGS. 1A-1B.

In operation, the data processing modules 106 and/or the third-party integrations 108 can receive one or more inputs (input sensor data 180a through 180n (e.g., 180a . . . 180n)), and can process the inputs and generate output data 190a through 190n (e.g., 190a . . . 190n). For example, the data processing modules 106 and/or the third-party integrations 108 can receive sensor data from electrical sensor 104a and the optical sensor 104b and can process the received data via algorithms 140, ML models 142, and/or monitoring engine 146 to generate a vasodilation estimation (and/or a vasoconstriction estimate) for display on an app 154. To carry out the processing, one or more data processing modules 106 can interact with one or more processors 114, memory 118, and/or sensors 104 to determine a vasodilation response, a vasoconstriction response, and/or related physiological responses. Monitoring and/or characterizing vasodilation responses can result in indications of biological changes, such as menopause, or anomalous biologic events, such as stroke, diabetes, peripheral blood circulation disorders, etc.

As shown in FIG. 1C, the data processing modules 106 include algorithms 140, optional ML models 142, user interface generator 144, monitoring engine 146, and apps 154. The monitoring engine 146 can further include an event detector 148, an alert generator 150, and an analysis module 152. In some implementations, the data processing modules 106 can be stored and executed as data processing modules 120 (FIG. 1B) on wearable device 102.

The algorithms 140 can include computer executable code adapted to carry out any of the methods described herein. In some implementations, the algorithms 140 utilize third party integrations 108 and/or optional ML models 142 to generate output. In operation, the algorithms 140 can operate on particular sensor data 180a . . . 180n (e.g., obtained from sensors 104 on device 102). The operations can prepare the data (e.g., preprocess) and/or otherwise analyze the data to generate estimations (e.g., PPG, respiration rate, heart rate, event occurrence, etc.). The data processing modules 106 can control operations of the device 102. For example, the data processing modules 106 can output activation signals to a stimulus source. In some implementations, the data processing modules 106 can additionally control operations of auxiliary devices (e.g., devices 500, 600, 650, 700, and/or 720) described herein. For example, the data processing modules 106 can output an activation signal to a stimulus source and/or one or more sensors of the auxiliary devices described herein to control a portion of such devices.

The optional ML models 142 can use machine learning techniques to estimate vasodilation activity for blood vessels associated with a particular skin surface site. For example, the ML models 142 can perform analysis, pattern classification, and/or recognition algorithms on PPG signals to assess changing optical properties of underlying tissues triggered by a stimulus provided by a wearable device. Portions of the analyzed signals can be used to generate vasodilation activity estimations. In some implementations, the ML models 142 can operate on signals obtained from sensors 104. The operations can include signal processing that can employ signal processing tools, for example filtering, extracting, digitizing, data de-convolution, machine learning, and/or other methods known in the art. Specifically, the signal processing can use higher order statistics to ascertain hidden patterns in data. Use of higher order statistics, known as cumulants, and their Fourier spectra, often termed poly spectra, can reveal the amplitude information in the higher order (such as those carried by power spectra or auto correlation) and can also include phase information. Phase information can reveal salient features of the data, otherwise unattainable from simple harmonic analysis.

The user interface generator 144 can interact with one or more processors 113, 114, memory 115, 118, and/or sensors 104. The user interface generator 144 can generate user interfaces for display to a user of the wearable device 102 or other user associated with the wearable device 102. The user interfaces can include patient data, sensor measurements and data, instructions, diagnosis data, or the like. The user interface can be presented on the display 112 of the wearable device and/or on a display of a companion device such as a mobile phone, laptop, tablet, or computer configured to receive information and user interfaces from the wearable device 102. The user interface can be configured to display a vasodilation response of the user over time, relative to a population, across multiple skin sites of a user, etc. The user interface can be further configured to display additional physiological responses of the user over time, relative to a population, across multiple skin sites, etc., for example an electrodermal activity response, muscle activity, hydration state, etc. The vasodilation response and physiological response(s) can be overlaid or displayed independently depending on the intended analysis. In some implementations, the user interface generator 144 can generate a user interface for device 102 for presenting data captured from an auxiliary device (e.g., case in communication with a wearable electronic device, such as device 102.

The monitoring engine 146 can determine when events or data changes occur at one or more of the sensors 104 (or associated electrodes). The monitoring engine 146 includes an event detector 148, an alert generator 150, and an analysis module 152. The event detector 148 can monitor and detect biological signals to monitor or characterize the signals or determine whether an anomalous biologic event has occurred. The analysis module 152 can function in combination with the event detector 148 to determine whether an event (or change in biological data) is to trigger an alert. The alert generator 150 can receive the determination of whether to trigger an alert and in response, can trigger the alert. For example, the temperature sensor 104g can function with the monitoring engine 146 to monitor and obtain temperature at a particular skin surface site associated with the wearable device 102. The monitoring engine 146 can trigger the alert generator 150 to generate an alert in response to determining that a monitored temperature has reached or exceeded a predefined threshold temperature.

In some implementations, the monitoring engine 146 can monitor aspects of an auxiliary device (e.g., devices 500, 600, 650, 700, and/or 800) that is in communication with the device 102. For example, the monitoring engine 146 can monitor signals generated by sensors on such auxiliary devices.

The alert generator 150 can alert the user wearing the wearable device 102 and/or alert a third party of an event, available data, and/or a change in data. In some implementations, the alert can be an audible sound or a visual indicator or message to the user via the device 102 or via a mobile device, computing device associated with the device 102. In some implementations, the alert can be a message sent to emergency services or physicians. Alerting emergency services or physicians, data including medical history can be transmitted directly to emergency services or physician computing systems, either directly from the wearable device 102 or from a remote memory, initiated by a signal from the wearable device 102. In addition to alerts, the wearable device 102 can also instruct a user to undertake or automatically activate certain treatments.

The apps 154 can represent one or more software applications that can be installed on (or accessed from) the wearable device 102 (and/or an associated mobile phone or computing device 107). The apps 154 can be used to present user interfaces generated by user interface generator 144.

Optionally (shown by dashed lines), the environment 101 can further include a third-party device or integration 108, for example a device including Amazon® Alexa® or an Amazon® Echo® device, as described in further detail elsewhere herein. For example, there can be bidirectional communication (e.g., via a wired connection or wireless communication) between the hardware component and the data processing modules 106, the data processing modules 106 and the third-party device or integration 108, and/or the third-party device or integration 108 and the hardware component.

Third party integrations 108 can optionally function with the wearable device 102, sensors 104, and/or data processing modules 106 to generate additional data for a user or medical practitioner. Third party integrations 108 can include third party services 160, third-party monitoring services 162, third party data services 164, mobile device 166, and apps 168.

Third party services 160 can provide monitoring services 162 and/or data services 164 to device 102 via a Wi-Fi network, a cellular network, cloud networks, or the like. The third-party monitoring services 162 and/or data services 164 can be provided directly to the device 102 or indirectly to device 102 via a mobile device 166. In some implementations, the third-party monitoring services 162 and/or data services 164 can be provided indirectly to the wearable device 102 via apps 168 that can interact with sensor data and device 102.

In some implementations, the data processing modules 106 and algorithms 140 described herein can be executed on wearable device 102. In some implementations, the data processing modules 106 and algorithms 140 described herein can be executed on the system 100. In some implementations, the data processing modules 106 and algorithms 140 described herein can be executed on environment 101. In some implementations, the data processing modules 106 and algorithms 140 described herein can be executed on computing devices (e.g., computing devices 107) or third-party integrations (e.g., third-party integrations 108). In some implementations, the data processing modules 106 and algorithms 140 described herein can be executed in a distributed fashion across one or more of wearable device 102, system 100, environment 101, computing device 107, and/or third-party integrations 108.

Although not depicted in FIGS. 1A-1C, a network can enable communications amongst wearable device 102, sensors 104, data processing modules 106, and/or third-party integrations 108. The network can also be configured through communication module 110 to enable communications with computing devices that are configured to communicate with device 102.

### Example Biological Characterization Process

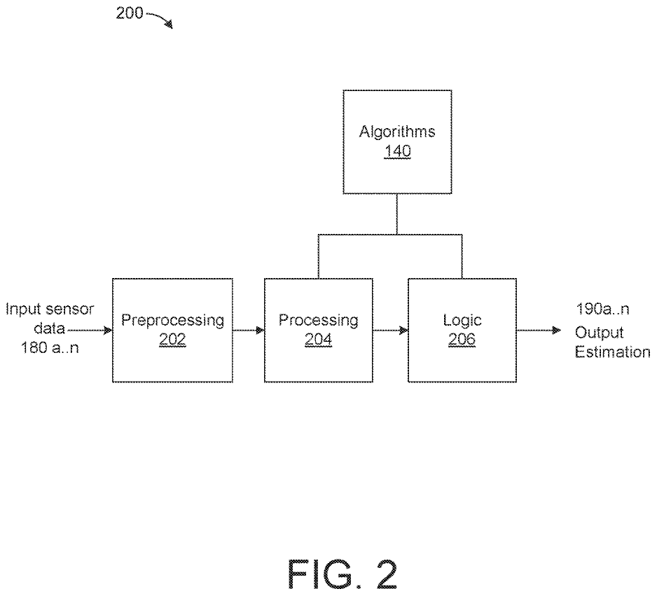

FIG. 2 illustrates a flow diagram of an example process 200 for generating biological data characterizations and/or estimates. The process 200 can be implemented by any of the devices and/or systems discussed above. The process 200 can include the steps of obtaining sensor data corresponding to at least one of inputs 180a through 180n (e.g., 180a . . . 180n), preprocessing (202) at last a portion of the sensor data, processing (204) the preprocessed data, and performing (206) logic to characterize vasodilation of one or more skin surface sites. In some implementations, the processing block 204 and/or logic block 206 can include any or all steps associated with algorithms 140.

For example, an optical sensor input 104b can be received as input 180b into preprocessing block 202. The preprocessing block 202 can perform signal preparation, signal normalization, and/or noise reduction before providing a preprocessed signal to processor block 204. Processing block 204 can use one or more algorithms 140 to perform analysis to generate a vasodilation estimation based on the original signal data 180a . . . n. A processed signal can be provided to logic block 206 to undergo comparisons, and/or further signal analysis. In some implementations, the processing block 204 and the logic block 206 can function to iteratively pass data back and forth to further process the signals and/or related data. A vasodilation estimate can be generated by logic block 206 upon meeting predefined criteria described in detail throughout this disclosure and in at least Appendix A (as incorporated above).

### Systems for Characterizing Bilateral Biological Response(s)

FIG. 3 illustrates a first wearable device 102a and a second wearable device 102b for measuring biological data and response asymmetry across a right and left limb, respectively. In this example, the device 102a is worn on a left limb 302 of a body of a person and the device 102b is worn on a right limb 304 of the body of the person. In some implementations, the wearable device 102a is positioned on a left limb or appendage (e.g., arm, leg, finger, hand, foot, toe, ear, etc.) or extremity of a person and a second device 102b is positioned on a right limb or appendage (e.g., arm, leg, finger, hand, foot, toe, ear, etc.) or extremity of the person. The device 102a and the device 102b can be in communication with the data processing modules 106 and third-party device or integration 108 as described herein.

The first and second devices 102a, 102b can measure similar parameters or features so that the parameters or features are comparable over time and/or on an event-by-event basis to detect biological signals, asymmetrical biologic responses, and/or deviation from a baseline (e.g., individualized or population based). For example, the data processing modules 106 can compare right side blood volume signals (e.g., in response to an application of heat) to left side blood volume signals (e.g., in response to application of heat) to determine whether an anomalous biologic event has occurred. In some implementations, the application of heat can be applied to a skin surface by both devices 102a, 102b to stimulate or alter biological signals that can later be analyzed to generate predictions, estimates, or treatment recommendations. Further, the stimulus signal delivery between the two devices can be synchronized and accordingly the signals acquired from a right extremity and a left extremity can also be substantially synchronized in time; and comparing the synchronized signals from the left extremity and the right extremity to determine whether the anomalous biologic event occurred. Although two devices and body portions are shown, one of skill in the art will appreciate that a single body portion or site can be monitored, characterized, or otherwise analyzed over time. The analysis can be in comparison to a baseline, a population-based level, historical data, or the like.

For example, the wearable device 102a can be applied to a skin surface site on a left extremity (e.g., left limb 302) and the wearable device 102b can be applied to a skin surface site on a right extremity (e.g., right limb 304). Both devices 102a, 102b can stimulate a response and measure the response from the left and right extremities, respectively. For example, the stimulus can be applied bilaterally (e.g., to detect asymmetrical responses) to each device 102a, 102b. The data processing modules 106 can receive signal data detected by sensors of devices 102a, 102b and can determine whether the responses or the difference in responses between the two sides indicates an atypical event, a stroke event, or a deviation from baseline.

### Example Wearable Device I

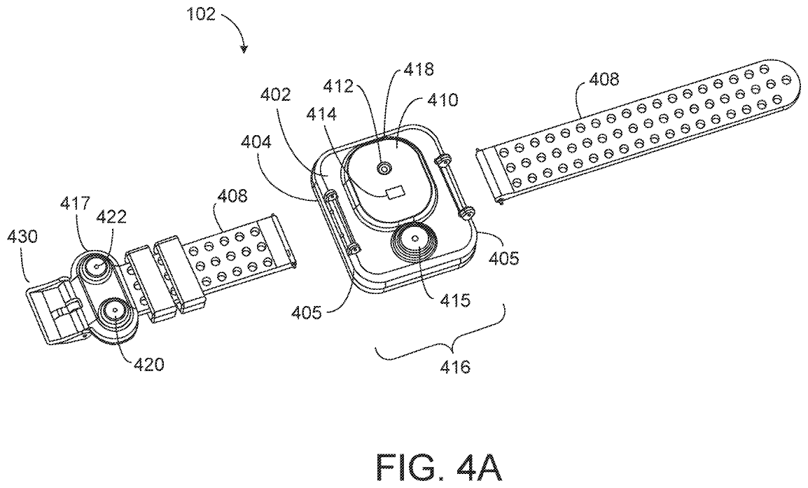

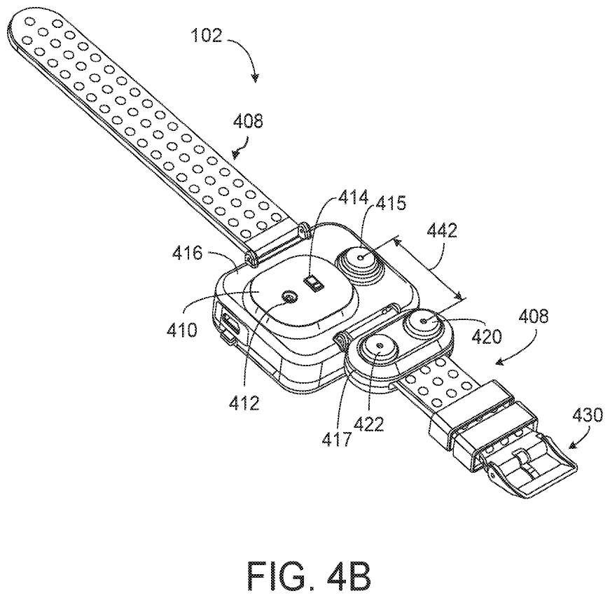

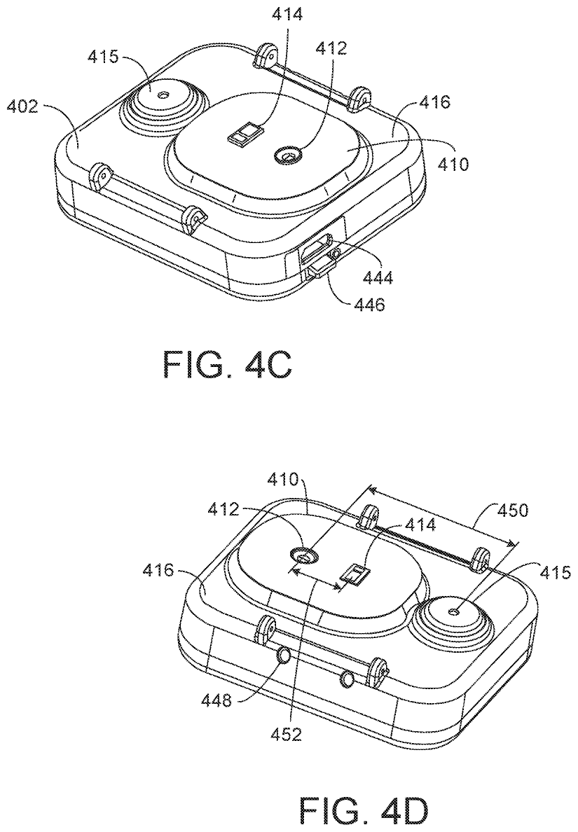

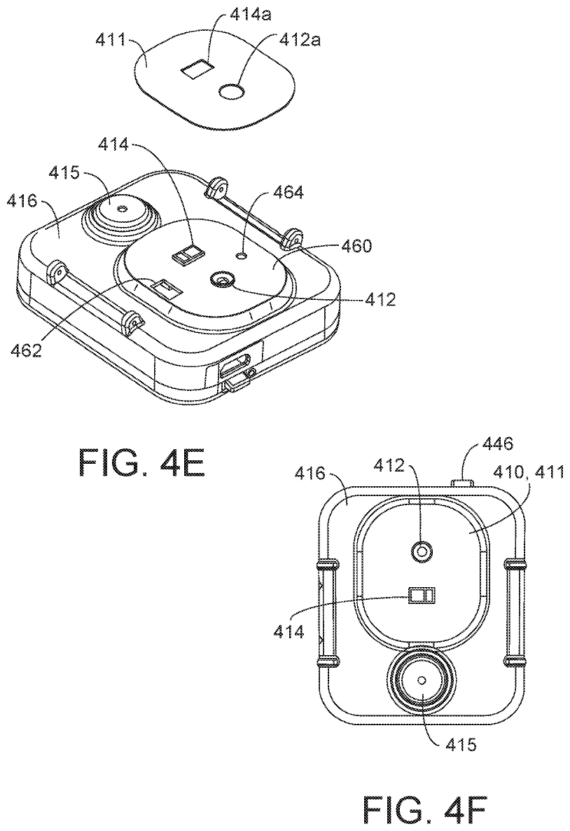

FIGS. 4A-4F illustrate a skin-facing side of an example device 102 configured to face a surface of a body (e.g., limb 302 or limb 304 of FIG. 3). As shown in FIG. 4A, the device 102 includes a body portion 416 having a first surface 404 opposite a second surface 402 configured to be in contact with a skin surface of a person. The first surface 404 and second surface 402 can be coupled via one or more or a plurality of sidewalls 405. For example, one or more sidewalls 405 can extend from a perimeter of the first surface 404 and couple to a perimeter of the second surface 402. The first surface 404 and/or second surface 402 can include one or more sensors positioned thereon. For example, one or more sensors on the first surface 404 can measure an environment of the person wearing or using the wearable device 102, and one or more sensors on the second surface 402 can measure one or more properties, features, or characteristics of the skin surface of the person. Alternatively, the first surface 404 can include one or more sensors or imagers or cameras for assessing a facial region of a person.

The wearable device 102 can include a stimulus source 410 (e.g., stimulus source 132 of FIG. 1B) in communication with the skin surface. In some implementations, a stimulus source 410 is positioned on a second surface 402 of the body portion 416, so that there is coupling or contact between the stimulus source 410 and a skin surface. Alternatively or additionally, a stimulus source 410 or one or more sensors (e.g., sensors 412, 414) and/or electrodes (e.g., electrode 415, 420, 422) can be positioned on a band 408 of the device 102, such that the body portion 416 is separate from a sensor module 418 (e.g., including one or more sensors 104, 412, 414, etc.) that includes the stimulus source 410 and the one or more sensors selected from sensors 104, for example. In some implementations, the stimulus source and/or one or more sensors can be distributed between the band, body, and sensor module depending on which sensors are incorporated into the system and their specific requirements or parameters.

The band 408 can be an adjustable or tensionable band including a buckle 430 on a first side and a series of receiving notches on a second, opposing side. Band 408 can allow for similar or alternative number and arrangements of electrodes 415, 420, and/or 422. In some implementations, a Velcro® or another hook-and-loop fastener (not shown), and/or a stretchable material (e.g., silicone, rubber, Lycra, Spandex, Elastane, neoprene, leather, fabric, etc.) can be employed along at least a portion of the band 408.

In some implementations, the band 408 can include connectors (not shown) for connecting electrodes (for example, electrodes 420 and 422) to other elements housed in the body portion 416 including, but not limited to, a power supply (for example, a battery) and a processor (for example, processor(s) 114 of FIG. 2). The connectors can be electrical traces, for example wires, conductive ink, circuitry or another connector, optical connectors, for example fiber optic cable, or other suitable connector that can be on, or at least partially embedded in a band 408. In some implementations, these traces from the electrodes 415, 420, and/or 422 of the band 408 to elements of the body portion 416 can be formed by cold molding or insert molding. In some implementations, connector wires can be threaded, woven, or sewn into the material of the band 408 and/or holes, channels, or other apertures in the band 408. In some implementations, the band 408 is at least partially made of a conductive material. In some implementations, connectors from a band 408 are connected to components (e.g., a battery, processor(s) 114) inside the body portion 416 through one or more holes (e.g., holes 448 of FIG. 4D) in the body portion 416.

In some implementations, the band 408 can be integrated into a clothing item, such as a shirt, a tight, a sock, pants, a shoe, a tongue of a shoe, laces of a shoe, sides of a shoe such that the device 102 is positioned on a particular region of a body portion of a user, for example.

The band 408 can carry an electrode housing 417 that can hold additional electrodes, such as two electrodes 420 and 422. The electrode housing 417 can be integral with or formed as a unitary structure with the buckle 430. In some implementations, the electrode housing 417 can be openable and/or removable from the band 408. The electrode housing 417 can include a lower portion and upper portion for housing one or more electrodes, for example electrodes 420 and 422. The lower portion and/or upper portion can include features for securely seating and retaining electrodes and/or an end of band 408 and/or a buckle 430 and/or an upper portion of the band. These securing features can include recesses, notches, alignment pins, fasteners, and/or clips to hold and/or align the assembly components. In some implementations, the electrode housing 417 is secured to a particular location on the band 408, where the electrode housing 417 cannot slide (for example, with pins and holes 448). In some implementations, the lower portion and upper portion securely mate together and cannot be opened. In some implementations, the lower portion and upper portion securely mate together and can be opened by manipulating the pieces, for example, by sliding or twisting the pieces against each other, and/or opening a latch, button, or other retention feature. In some implementations, the electrode housing 417 is secured to the band 408 and can slide along the length of the band. For example, the lower portion and upper portion can secure together over and/or around the band 408 and provide a clamping force to the band 408 to secure the electrode housing 417 in position while being slidable along the band 408. In some implementations, electrode housing 417 is adjustable or positionable along a length of the band 408 such that the electrode housing is positioned on a different region of a body portion than body portion 416.

Referring again to FIG. 4A, the body portion 416 further includes a blood volume sensor 412 and, optionally, a skin temperature sensor 414. The blood volume sensor 412 can be integrated into a form factor such as the device 102 that improves continuous anomalous cardiac event monitoring. The blood volume sensor 412 can measure vasodilation response parameters. The skin temperature sensor 414 can also be integrated into the device 102. The skin temperature sensor 414 is positioned on the second surface 402 and can measure a temperature of the skin surface in contact with the stimulus source 410. The blood volume sensor 412 is positioned on the second surface 402 and can measure a blood volume of the skin surface. The blood volume sensor can be a photoplethysmography sensor or an impedance plethysmographic sensor. The blood volume sensor can employ light at one or more of 450-500 nm (blue), 500-570 nm (green), 570-610 nm (yellow), 610-760 nm (red), or infra-red (>760 nm) wavelength, or a combination thereof. Different wavelengths can be more appropriate for different applications, for example green (500-570 nm) light can be more accurate for heart rate measurements (e.g., heart rate variability, heart rate, etc.). In addition to, or alternatively, the blood volume sensor can also measure one or more of: heart rate, heart rate variability, or oxygen saturation.

FIG. 4B illustrates a perspective view of the wearable device of FIG. 4A. A second surface 402 of a body portion 416 is depicted. The second surface 402 of the body portion 416 can be similar or identical to a second surface 402 of other devices described herein, for example second surface 402 can include a heat source 410, blood volume sensor 412, and skin temperature sensor 414. In some implementations, the second surface 402 includes one electrode 415. A band 408 with a buckle 430 can include an electrode housing 417 that can carry additional electrodes, such as two electrodes 420 and 422. The electrodes 420 and 422 (and/or other additional electrodes and/or sensors) can be positioned on the second surface 402 and/or a tensionable band 408 of the device 102. The electrodes 415, 420, and/or 422 can be located away from the surface of the device 102. For example, electrode 415 can be placed on a raised platform 460 of the second surface 402 of the body portion 416. As discussed above with regard to electrodermal sensors 412 and 414, electrodes 415, 420, and/or 422 can be spaced at preselected distances. In some implementations electrodes 420 and 422 are spaced at a distance of about 5 mm to about 100 mm, for example 5 mm to about 10 mm, about 10 mm to about 20 mm, about 20 mm to about 30 mm, about 30 mm to about 40 mm, about 40 mm to about 50 mm, about 50 mm to about 60 mm, about 60 mm to about 70 mm, about 70 mm to about 80 mm, about 80 mm to about 90 mm, about 90 mm to about 100 mm, measured from a center point of each electrode 420, 422.

In some implementations, electrodes 420 and 422 can be spaced apart from the stimulus source 410 and/or electrode 415 by a distance 442, which can be about 10 mm to about 300 mm, for example about 10 mm to about 20 mm, about 20 mm to about 30 mm, about 30 mm to about 40 mm, about 40 mm to about 50 mm, about 50 mm to about 60 mm, about 60 mm to about 80 mm, about 80 mm to about 100 mm, about 100 mm to about 120 mm, about 120 mm to about 150 mm, about 150 mm to about 175 mm, about 175 mm to about 200 mm, about 200 to about 225 mm, about 225 mm to about 250 mm, about 250 mm to about 275 mm, about 275 mm to about 300 mm, measured from a center point of the electrodes 420, 422 to a center point of the heat source 410 and/or the electrode 415. Still further, electrode 415 can be spaced apart from a heat source 410 by a distance which can be about 10 mm to about 20 mm, about 20 mm to about 30 mm, about 30 mm to about 40 mm, about 40 mm to about 50 mm, about 50 mm to about 60 mm, about 60 mm to about 70 mm, about 70 mm to about 80 mm, about 80 mm to about 90 mm, about 90 mm to about 100 mm, measured from a center point of the electrode 415 and a center point of the heat source 410. When the body portion 416 is secured to the wrist with the band 408, the electrodes 420 and 422 can be positioned on the band such that they contact the skin approximately along a midline of palm side of the wrist. In such examples, the electrode 415 can be positioned approximately on a midline on the dorsal side of the wrist.

FIG. 4C illustrates a partial view of the wearable device 102 of FIG. 4A. As shown, the body portion 416 of the device 102 includes a port 444 for electrically coupling the device 102 to a power source, for example to charge a battery (such as battery power 133) in the device 102. Additionally or alternatively, port 444 can electrically couple the wearable device to an external or remote computing device (e.g., laptop, desktop, server, workstation, etc.) to download data from the device or upload system parameters or install updates to the wearable device 102. Port 444 can also be used to connect auxiliary sensors, an input/output device (keyboard, joystick, buttons, switches, printer, camera, display), and/or memory unit. The wearable device 102 can further include one or more user interface elements 446, for example one or more buttons and/or switches, that can be used, for example, to power on and off the device, to input user specific reactions, features, or characteristics, to customize an interface or functionality of the user device, to mark events, to initiate pairing or data transfer, to call for help, etc. User interface elements 446 can alternatively or additionally include output and/or feedback elements, such as a speaker, light, and/or haptic stimulator. In some implementations, user interface element 446 can be used, for example, to indicate power on, charging, low battery, pairing mode, heating phase, malfunction, health event, and/or other status of the device 102 and/or user. In some implementations, user interface element 446 can be a feedback element that includes one or more LED behind a smoked, translucent, or transparent window. In some implementations, there is no display screen on the body portion 416.

FIG. 4D illustrates a rotated view of the wearable device 102 of FIG. 4C. As shown, the second surface 402 of body portion 416 can include a stimulus source 410, blood volume sensor 412, skin temperature sensor 414, and electrode 415, as discussed above. The second surface 402 of body portion 416 can be arranged to contact the dorsal or top side of the foot when worn to locate the stimulus source 410, blood volume sensor 412, skin temperature sensor 414, and electrode 415 generally along a midline of the top or dorsal side of the foot. Alternatively, the second surface 402 of body portion 416 can be arranged to contact the sole or bottom side of the foot when worn to locate the stimulus source 410, blood volume sensor 412, skin temperature sensor 414, and electrode 415 generally along a midline of the sole or bottom side of the foot. Blood volume sensor 412 and skin temperature sensor 414 can be placed within the stimulus source 410 spaced at a distance 452 from each other. Distance 452 can be about 10 mm to about 100 mm, for example 10 mm to about 20 mm, about 20 mm to about 30 mm, about 30 mm to about 40 mm, about 40 mm to about 50 mm, about 50 mm to about 60 mm, about 60 mm to about 70 mm, about 70 mm to about 80 mm, about 80 mm to about 90 mm, about 90 mm to about 100 mm, measured from a center point of the blood volume sensor 412 and a center point of the skin temperature sensor 414. Similarly, blood volume sensor 412 can be placed at a distance 450 from the electrode 415. Distance 450 can be about 10 mm to about 200 mm, for example 10 mm to about 20 mm, about 20 mm to about 30 mm, about 30 mm to about 40 mm, about 40 mm to about 50 mm, about 50 mm to about 60 mm, about 60 mm to about 70 mm, about 70 mm to about 80 mm, about 80 mm to about 90 mm, about 90 mm to about 100 mm, about 100 mm to about 120 mm, about 120 mm to about 140 mm, about 140 mm to about 160 mm, about 160 mm to about 180 mm, about 180 mm to about 200 mm, measured from a center point of the blood volume sensor 412 and a center point of the electrode 415.

FIG. 4E illustrates a perspective view and an exploded view of the wearable device 102 of FIG. 4C. As shown, the second surface 402 of the body portion 416 can include a raised platform 460. Platform 460 can include the stimulus source 410, blood volume sensor 412, and skin temperature sensor 414. In some implementations, the platform 460 can include electrode 415. Platform 460 can improve contact between the skin and the heat source 410, blood volume sensor 412, and skin temperature sensor 414. In some implementations, the platform 460 is sized to cover or substantially cover a portion of a dorsal region of a foot, a portion of a sole of a foot, or a portion of an ankle when device 102 is worn. In some implementations, the platform 460 is flexible and/or shaped, for example, curved, and can increase a contact area between the platform 460 and the skin when the device 102 is worn. In some implementations, stimulus source 410 symmetrically surrounds the blood volume sensor 412 and/or symmetrically surrounds the skin temperature sensor 414. In some implementations, stimulus source 410 can surround the blood volume sensor 412 with an area approximately equal to the area of stimulus source 410 that surrounds the skin temperature sensor 414.

In some implementations, the stimulus source 410 can include a warming plate 411, as shown in FIG. 4E. For example, the stimulus source 410 can include a warming plate 411 for increased heat distribution and/or heat retention. The second surface 402 of body portion 416 can include an opening, such as opening 462, to allow the stimulus source 410 and/or warming plate 411 to communicate with and/or access other components inside the body portion 416, for example the processor(s) 114 and battery power 133. The second surface 402 of the body portion 416 can include a thermistor 464 or other temperature sensor for monitoring the temperature of the stimulus source 410 and/or warming plate 411. The thermistor 464 can provide a heater temperature measurement, which can be used to control the stimulus source 410.

Warming plate 411 can include apertures for components surrounded and/or enclosed by the heat source 410, for example aperture 412a for blood volume sensor 412 and aperture 414a for skin temperature sensor 414. Aperture 412a and aperture 414a can allow improved contact between the skin and blood volume sensor 412 and skin temperature sensor 414. In some implementations, the blood volume sensor 412 includes two separate components, for example an emitter and a detector, and accordingly aperture 412a would include an aperture for each component. In general, heat source 410 can be a layered or laminate structure. In some implementations, the heat source 410 can include dimples and/or perforations for heat distribution and/or dissipation. Perforations can also improve adhesion.

FIG. 4F illustrates a front view of the wearable device 102 of FIG. 4C. The warming plate 411 is shown installed on heat source 410 of wearable device 102. Alternative or additional features, such as ridges, channels, fins, and the like can also be included on a heat source 410 to improve uniform heating and cooling and/or direct heating and cooling. For example, surface features can be used to direct heat from the heat source 410 toward or away from a lower extremity facing side, an ankle facing side, a foot facing side, or skin facing side of the device 102. The surface features can alternatively, or additionally, be located on a warming plate 411. Heat source 410 can be positioned on a raised platform 460 of the second surface 402 of the body portion 416. In some implementations, the heat source 410 is arranged farther from the hand than the electrode 415 when the device 102 is worn on the lower extremity.

### Example Auxiliary Devices

FIG. 5A illustrates a perspective view of an example implementation of an electronic auxiliary case 500 for obtaining biological data. The case 500 can represent a system for receiving and retaining a wearable electronic device. For example, the case 500 can be a housing (e.g., skin, protective case, film, encasement, etc.) for a wearable electronic device, such as wearable device 102, wearable electronic device 530, or another electronic device that can be at least partially encased within case 500. In some implementations, the case 500 can be rigid with one or more tension fit surfaces to retain (e.g., hold) a wearable electronic device. In some implementations, the case 500 can be flexible with one or more flexible surfaces to surround and retain portions of a wearable electronic device. In some implementations, the case 500 can include both rigid portions and flexible portions to retain a wearable electronic device.

The case 500 can have an open end 502 defined by an edge perimeter 501 effective for retaining a first portion (e.g., a face of surface B of the wearable device 102) or a face of another wearable electronic device. The case can also include a base 506 for retaining a second portion (e.g., a surface A of the wearable device 102) or another wearable electronic device. In some implementations, a wearable electronic device can be at least partially enclosed within case 500 such that at least a portion of a top surface (e.g., surface B in FIG. 1A of device 102) is surrounded on the edge perimeter 501 around an opening 502. The opening 502 can allow for a user to view and interact with a face of surface B of device 102 (or a face of another wearable electronic device) without being encumbered by the case 500. The case 500 can also partially encase device 102 while allowing at least a portion of a bottom surface (e.g., surface A of device 102 or a rear surface of another wearable electronic device) to be open for access at an opening 504 formed in a base 506 of case 500. The opening 504 can allow for a user to have access to particular sensors or accessories on a rear surface of the wearable electronic device without having to remove the case 500.

The case 500 can also surround other surfaces of a wearable electronic device, as shown by sidewall 508, sidewall 510, sidewall 512, and sidewall 514 and corners 519 that can connect each adjacent sidewall. The sidewall 508 is depicted opposite a sidewall 514. The sidewall 510 is depicted opposite a sidewall 512. In some implementations, each sidewall 508-514 can be configured to couple to the base 506. In some implementations, each sidewall 508-514 can extend away from the base 506 to form sides of the case 500. In such an example, the sidewalls 508-514 and base 506 are composed of a single seamless material from the base 506 to the edge perimeter 501 of the case 500. Although four sidewalls are shown in a substantially square structure, one of skill in the art will appreciate that any number of sidewalls and/or shapes/structures are contemplated herein.

In some implementations, the case 500 can be a housing for retaining a wearable electronic device 530. The case 500 can include a flexible housing defining the open end 502 opposite the base 506. The open end 502 can be further defined by the perimeter 501 that is effective for retaining the first portion 530a of the wearable electronic device 530. The base can retain the second portion 530b of the wearable electronic device 530. The case 500 can further include the stimulus source 516. The stimulus source 516 can include a surface area extending on a first portion of a surface of the base and through the first opening 504 defined by the surface of the base 506. The case 500 can also include at least one sensor 518 positioned on a second portion of the surface of the base 506 and in a second opening (not shown) defined by the surface of the base 506. The stimulus source 516 can be in electrical contact with the at least one sensor 518.

The case 500 can also include a stimulus source 516. The stimulus source 516 can include a surface area (e.g., length (l)*width (w), as shown in FIG. 5A) extending on a first portion of the surface of the base 506 and through the opening 520 (FIG. 5C) in the base 506. The opening 520 can allow the stimulus source 516 to be placed in contact with a body site of a person wearing the wearable electronic device with the case 500 in order to warm or cool the body site.

Example surface areas that the stimulus source 516 can occupy can be about 500 mm² to about 700 mm²; about 550 mm² to about 600 mm²; about 600 mm² to about 650 mm²; about 650 mm² to about 700 mm². Example width (w) can be about 15 mm to about 30 mm; about 15 mm to about 20 mm; about 20 mm to about 25 mm; about 25 mm to about 30 mm. Example lengths (l) can be about 20 mm to about 35 mm; about 20 mm to about 25 mm; about 25 mm to about 30 mm; about 30 mm to about 35 mm. In some implementations, the stimulus source 516 is sized to enclose at least one skin temperature sensor and at least one optical sensor.

In some implementations, the stimulus source 516 can function in the same manner as stimulus source 410 on device 102 to emit heat or coolness. However, stimulus source 516 is instead installed in the case 500 (e.g., an auxiliary device) rather than the wearable electronic device 102. The stimulus source 516 can function with one or more sensors on a particular wearable electronic device when such a device is communicatively coupled to case 500, for example.

The case 500 can also include at least one sensor 518. The sensor 518 can be positioned on a second portion of the surface of the base 506. The sensor 518 can be installed within an opening 522 (FIG. 5C) on the surface of the base 506. The thermal stimulus can be in electrical contact with the sensor 518 (or any other sensor installed within case 500 and/or installed within the device being encased by case 500.

The sensor 518 can include or represent any number of sensors including any or all of sensors 104. In some implementations, the sensor 518 can be a skin temperature sensor, such as temperature sensor 104g, as described in detail herein. In some implementations, the sensor 518 can be an optical PPG sensor 104b, as described in detail herein. In some implementations, additional sensors can be included and installed within case 500 including, but not limited to an electroencephalogram (EEG) sensor, an inertial measurement unit (IMU) sensor, a heart rate (HR) sensor, an electrodermal activity (EDA) sensor, an electrocardiogram (ECG) sensor, and an electromyography (EMG) sensor, mechanical sensors, a pressure sensor, a motion sensor, a blood volume sensor, a muscle activity sensor, a sweat sensor, a tissue impedance sensor, or an environmental temperature sensor.

In some implementations, the sensor 518 and/or stimulus source 516 can add (or supplement) functionality to one or more sensors 104 of a wearable device that is encased within case 500. For example, the sensor 518 can be a temperature sensor while the sensor 104 of the wearable device 102, for example, can be a PPG sensor. The PPG sensor of case 500 can function with the temperature sensor of device 102 to generate additional parameters for the wearer of device 102 (paired to or coupled to device 500). The combination use of both sensors 518, 104 (or additional sensors) can be accomplished by programming the processor 113 of device 102 and/or the processor (e.g., processor 930) of an accessory/auxiliary device (e.g., device 902, 530, etc.) to allow the sensors 518, 104 to interact and to allow one or both device processors, 930, 113, etc. to perform calculations, determinations, or the like, as described in detail herein. Similarly, when an accessory/auxiliary case 500 is paired/coupled to a main wearable electronic device 530, a stimulus source (e.g., stimulus source 516) of the case 500 can be used to invoke particular responses from a user wearing the paired/coupled devices. For example, the stimulus source 516 can generate a thermal stimulus that is applied or radiated at a body site of the user. A sensor 104 can be a PPG sensor for detecting PPG signals in response to the applied thermal stimulus. The sensor 104 can obtain the PPG signals and provide such signals (or data based on the signals) to the processor 930 of the case 500. The processor 930 of case 500 can determine any number of vasodilation estimates based on the PPG sensed data obtained from the device 530 (responsive to triggered application of the thermal stimulus provided by stimulus source 516). In some implementations, the sensor 518 can be an auxiliary sensor for interpreting sensor signals from the sensors 104 on the encased wearable electronic device (e.g., device 530, device 102, etc.).

As shown in FIG. 5C, the base 506 can further include at least one connection port 524 configured to electrically connect the sensor 518 and/or the stimulus source 516 to a receiving port (e.g., such as user interface port 446) when the wearable electronic device that is installed into case 500 includes the device 102. For example, the receiving port can be connected via wires 526 to the sensor 518 and/or the stimulus source 516. In some implementations, the receiving port 524 is coupled to an electronics module 528 for controlling and/or powering case components (e.g., stimulus source 516 and sensor 518, etc.) and communicating between the case 500 and the wearable electronic device. One or more connection terminals 538 can be included on case 500 that can interface with connection terminal 540 of device 530.

In some implementations, the case 500 can include a securing means or a band (not shown) to assist in wearing the case 500 that is housing a wearable electronic device 530. The band can be adjustable, stretchable, and/or tensionable. In some implementations, the case 500 can include a securing means comprising Velcro® or another hook-and-loop fastener (not shown), and/or a stretchable material (e.g., silicone, rubber, Lycra, Spandex, Elastane, neoprene, leather, fabric, etc.).

FIG. 5B illustrates a side perspective view of the electronic auxiliary case 500 of FIG. 5A. In this example, the case 500 is at least partially encasing wearable electronic device 530. A face surface 530a is viewable and accessible to a user because the case 500 does not cover or otherwise obstruct the face surface 530a when device 530 is installed within case 500. A rear surface 530b of device 530 is accessible to perform physiological skin measurements and assessments because the case 500 has an opening 504 to allow at least a portion of device 530 to be accessible when device 530 is installed within case 500. Although the opening 504 is shown arched, any shape can be contemplated.

In some implementations, the case 500 can represent a main (or master) device for a particular purpose while the wearable electronic device 530 enclosed within case 500 represents an auxiliary device. For example, the case 500 can represent a health or physiological system monitor while wearable electronic device 530 can represent one or more of a sensor patch, a watch, a smart watch, a fitness tracker, a phone, a wearable battery, a camera, a bracelet, a ring, a controller, and smart glasses. Each device 500, 530 can provide separate and/or combined functions and hardware to enable a user to obtain data about physiological changes and/or user activity.

In some implementations, the case 500 includes one or more batteries to operate the electronics and sensors installed within case 500. In some implementations, the case 500 is powered by a power source on the wearable electronic device 530, for example, when device 530 is installed within case 500.

In some implementations, the case 500 includes one or more control buttons (not shown). For example, the case 500 can include a control button to power up or power down the case to energize a battery installed within the case 500. In some implementations, the case 500 can include a control button to synchronize or otherwise connect the components of case 500 to a wearable electronic device installed within the case. In some implementations, the case 500 can include a control button to begin sensing physiological or environmental parameters surrounding a person wearing the case 500 and/or device within the case 500.

FIG. 5D illustrates a perspective view of an example implementation of another electronic auxiliary case 550 for obtaining biological data. The case 550 can be similar to case 500 of FIGS. 5A-5C, however, case 550 can include a stimulus source 552 that at least partially surrounds the opening 504. In the configuration of FIG. 5D, the stimulus source 552 can be arranged in the case 550 to surround (or at least partially surround) a body site in which to heat or cool. The case 550 can be placed in contact with the body site to ensure that the stimulus source 552 is arranged around a perimeter (or partial perimeter) of the body site. Measurements and/or signals can be obtained from sensor 518 responsive to heating or cooling the body site with stimulus source 552.

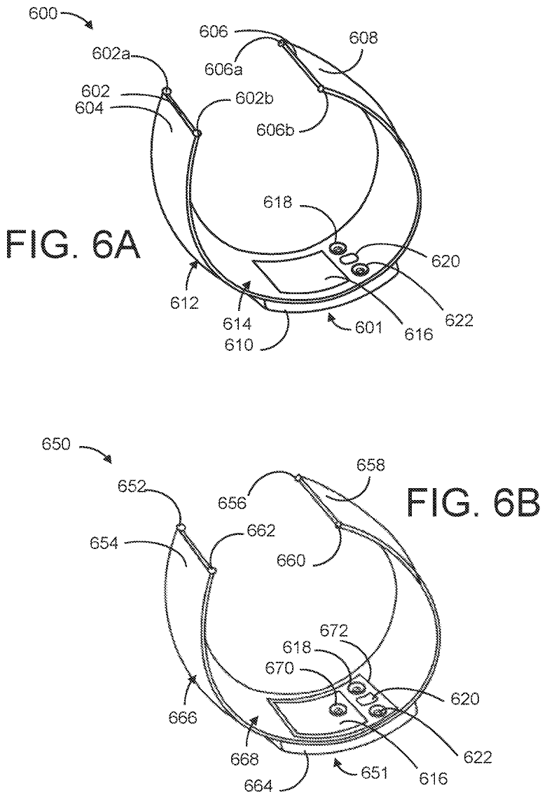

FIG. 6A illustrates a perspective view of an example implementation of an electronic band 600 for obtaining biological data. The electronic band 600 can function as a data collection system (and/or an accessory system) that uses a sensor system 601 to measure and analyze physiological parameters of a person wearing the band 600. In some implementations, the electronic band 600 can utilize and analyze data captured by sensors of a device that is coupled to the band 600.

The band 600 can be configured to be removably coupled to a wearable electronic device such as device 530, device 102, or another electronic device. For example, the band 600 can include one or more coupling parts to removably connect the wearable electronic device to the band 600 and/or to couple to other components installed within the band 600. For example, a first coupling part 602 can be provided on a first end 604 of the electronic band 600. A second coupling part 606 can be provided on a second end 608 of the electronic band 600. The first coupling part 602 or the second coupling part 606 can electrically couple to a connection terminal (e.g., connection terminal 540) of the wearable electronic device 530, for example.

One or more of the first coupling part 602 and the second coupling part 606 can removably couple the electronic band 600 to the wearable electronic device (e.g., device 530 or another electronic device) to cause a communicative connection between the sensor system 601 of the band 600 and the wearable electronic device that is removably coupled to the band 600. For example, coupling the wearable electronic device to the band 600 can include fastening one or more of the coupling parts 602, 606 to a portion of the wearable electronic device. The fastening mode can include snaps, electrical fittings, conduit bodies, locknuts, bushings, hubs, or other coupler. Although two coupling parts 602, 606 are depicted in FIG. 6A, any number of coupling parts can be used to communicatively and electrically couple band 600 to another electronic device.

In some implementations, the first coupling part 602 includes a first connector 602a and a second connector 602b. The first connector 602a can be in line with the second connector 602b. For example, the first connector 602a can be arranged parallel to and opposite the second connector 602b. The second coupling part 606 can include a third connector 606a and a fourth connector 606b. The third connector 606a can be in line with the fourth connector 606b. For example, the third connector 606a can be arranged parallel to and opposite the fourth connector 606b. Any one of the first connector 602a, the second connector 602b, the third connector 606a, and the fourth connector 606b is configured with connector circuitry for connecting to at least one connection terminal (e.g., connection terminal 540) of the wearable electronic device 530. For example, the at least one connection terminal causes the electronic band 600 to be in electrical communication with the wearable electronic device 530 when the respective connector 602a, 602b, 606a, or 606b is coupled to the at least one connection terminal 540 of the wearable electronic device 530.

The band 600 can be coupled to an electronics module 610 that is part of the sensor system 601. The electronics module 610 can be provided on an external surface 612 of the electronic band 600. The electronics module 610 can include power and electronic circuits for operating a number of sensors in the sensor system 601. The sensor system 601 can be configured to be communicatively coupled to the wearable electronic device (e.g., device 530, device 102, etc.) when the wearable electronic device is connected to the electronic band 600. In some implementations, the sensor system 601 can be at least partially in contact with a portion of an internal surface of the electronic band 600. The internal surface 614 can be opposite the external surface 612. Sensors of sensor system 601 can be at least partially installed through a first opening (not shown) in the electronic band 600 such that the sensors can be in contact with a body portion or in contact with an environment surrounding the body portion.

As shown in FIGS. 6A-6B, the first opening (not shown) is shaped to house a stimulus source 616 from the external surface 612 side to the internal surface 614 side. The stimulus source 616 includes a first surface area extending through a second opening (not shown) of the electronic band 600 to be positioned within the internal surface 614 of the band 600.

The sensor system 601 can include any number of sensors including, but not limited to an electroencephalogram (EEG) sensor, an inertial measurement unit (IMU) sensor, a heart rate (HR) sensor, an electrodermal activity (EDA) sensor, an electrocardiogram (ECG) sensor, and an electromyography (EMG) sensor, mechanical sensors, a pressure sensor, a motion sensor, a blood volume sensor, a muscle activity sensor, a sweat sensor, a tissue impedance sensor, or an environmental temperature sensor. As shown in FIG. 6A, the sensor system 601 includes a PPG sensor 618, a skin temperature sensor 620, and an electrode 622. In this example, the sensors 618-622 are arranged in parallel to each other and adjacent to the stimulus source 616. Other arrangements are possible. In addition, any number of sensors and/or electrodes can be included on band 600.

The sensor system 601 is depicted in FIG. 6A in a configuration that can be placed in contact with an underside of a wrist or ventral side of a wrist of a person. However, the sensor system 601 and/or the electronics module 610 can instead be positioned to be in contact with a lateral side of the wrist or a dorsal side of the wrist. In some implementations, the sensors 618-622 and the stimulus source 616 of the band 600 can be arranged to contact other body parts including, but not limited to, a finger, an arm, a leg, a foot, a forehead, a pulse point, etc.

In some implementations, the band 600 can include wiring (not shown) and/or circuitry (not shown) to couple the sensors 618-622 and/or the stimulus source 616 electrically and communicatively to an electronic device (e.g., wearable electronic device 530) that is physically fastened to the band 600 via coupling parts 602, 606. Such a fastening can also function to connect electrical wires and/or circuits in the band 600 to pins, ports, snaps, and/or receiving couplers on the electronic device to couple sensors 618-622 and/or the stimulus source 616 electrically and communicatively to sensors and/or components of the electronic device, such as device 530. The band 600 can be adjustable, stretchable, and/or tensionable.

FIG. 6B illustrates a perspective view of another example implementation of an electronic band 650 for obtaining biological data. The electronic band 650 can function as a data collection system (and/or an accessory system) that uses a sensor system 651 to measure and analyze physiological parameters of a person wearing the band 650. In some implementations, the electronic band 650 can utilize and analyze data captured by sensors of a device that is coupled to the band 650.

The band 650 can be removably coupled to a wearable electronic device such as device 530, device 102, or another electronic device. For example, the band 650 can include one or more coupling parts to removably connect the wearable electronic device to the band 650 and/or to couple to other components installed within the band 650. For example, a first coupling part 652 can be provided on a first end 654 of the electronic band 650. A second coupling part 656 can be provided on a second end 658 of the electronic band 650. A third coupling part 660 can be provided on the second end 658 across the band 650 from coupling part 656. A fourth coupling part 662 can be provided on the first end across from coupling part 652.

One or more of the first coupling part 652, the second coupling part 656, the third coupling part 660, or the fourth coupling part 662 can be removably coupled to the electronic band 650 to the wearable electronic device (e.g., device 530 or another electronic device) to cause a communicative connection between the sensor system 651 of the band 650 and the wearable electronic device that is removably coupled to the band 650. For example, coupling the wearable electronic device to the band 650 can include fastening one or more of the coupling parts 652, 656, 660, or 662 to a portion of the wearable electronic device. The fastening mode can include snaps, electrical fittings, conduit bodies, locknuts, bushings, hubs, or other coupler.

The band 650 can be coupled to an electronics module 664 that is part of the sensor system 651. The electronics module 664 can be provided on an external surface 666 of the electronic band 650, similar to band 600. The electronics module 664 can include power and electronic circuits for operating a number of sensors in the sensor system 651. The sensor system 651 can be communicatively coupled to the wearable electronic device (e.g., device 530, device 102, etc.) when the wearable electronic device is connected to the electronic band 650.

Similar to band 600, the sensor system 651 can be at least partially in contact with a portion of an internal surface 668 of the electronic band 650. The internal surface 668 can be opposite the external surface 666. Sensors of sensor system 651 can be at least partially installed through a first opening (not shown) in the electronic band 600.

The sensor system 651 can include any number of sensors including, but not limited to an electroencephalogram (EEG) sensor, an inertial measurement unit (IMU) sensor, a heart rate (HR) sensor, an electrodermal activity (EDA) sensor, an electrocardiogram (ECG) sensor, and an electromyography (EMG) sensor, mechanical sensors, a pressure sensor, a motion sensor, a blood volume sensor, a muscle activity sensor, a sweat sensor, a tissue impedance sensor, or an environmental temperature sensor. As shown in FIG. 6B, the sensor system 651 includes a PPG sensor 618, a skin temperature sensor 620, an electrode 622, and an electrode 670. In this example, the sensors 618-622 are arranged in parallel to each other and adjacent to the stimulus source 616. The electrode 670 is arranged within the surface perimeter of the stimulus source 616. In particular, the stimulus source 616 can surround the electrode 670.

In some implementations, the stimulus source 616 can be expandable along a width and/or length of the band 650. For example, a stimulus source 672 is depicted in FIG. 6B with an increased surface area as compared to stimulus source 616. The surface area of the stimulus source 672 can be from about 5 percent to about 40 percent larger than the surface area of stimulus source 616. For example, the difference in surface area between the stimulus source 616 and the stimulus source 672 can be about 5 percent to about 10 percent; about 10 percent to about 15 percent; about 15 percent to about 20 percent; about 20 percent to about 25 percent; about 25 percent to about 30 percent; about 30 percent to about 35 percent; about 35 percent to about 40 percent.

The stimulus source 672 surrounds the stimulus source 616, the electrode 670, the PPG sensor 618, the skin temperature sensor 620, and the electrode 622. In operation, the stimulus source 616 and/or the stimulus source 672 can function together as a single unit to thermally impact a body site. In some implementations, the stimulus source 616 and/or the stimulus source 672 can function separately to each thermally impact different portions of a body site. The stimulus source 616 and/or the stimulus source 672 can be operated over predefined time intervals that can or cannot overlap.

In some implementations, stimulus source 672 symmetrically surrounds the electrode 670. Other arrangements of the stimulus source 616, the stimulus source 672, the PPG sensor 618, the skin temperature sensor 620, the electrode 622, and the electrode 670 are of course possible.

Similar to band 600, the sensor system 651 in FIG. 6B is depicted in a configuration that can be placed in contact with an underside or ventral side of a wrist of a person. However, the sensor system 651 and/or the electronics module 664 can instead be positioned to be in contact with a lateral side or dorsal side of the wrist. In some implementations, the sensors 618-622, the electrode 670, the stimulus source 616, and stimulus source 672 of the band 650 can be arranged to contact other body parts including, but not limited to, a finger, an arm, a leg, a foot, a forehead, a pulse point, etc. The band 650 can be adjustable, stretchable, and/or tensionable to fit a body part.

In some implementations, the band 650 can include one or more components of FIGS. 1B, 1C, and 9 and/or additional wiring (not shown) and/or circuitry (not shown) to couple the sensors 618-622, the electrode 670, the stimulus source 616, and the stimulus source 672 electrically and communicatively to an electronic device (e.g., wearable electronic device 530) that is physically fastened to the band 650 via coupling parts 652, 656, 660 and/or 662. Such a fastening can also function to connect electrical wires and/or circuits in the band 650 to pins, ports, snaps, and/or receiving couplers (or port) on the electronic device to couple the sensors 618-622, the electrode 670, the stimulus source 616, and the stimulus source 672 electrically and communicatively to sensors and/or components of the electronic device, such as device 530.

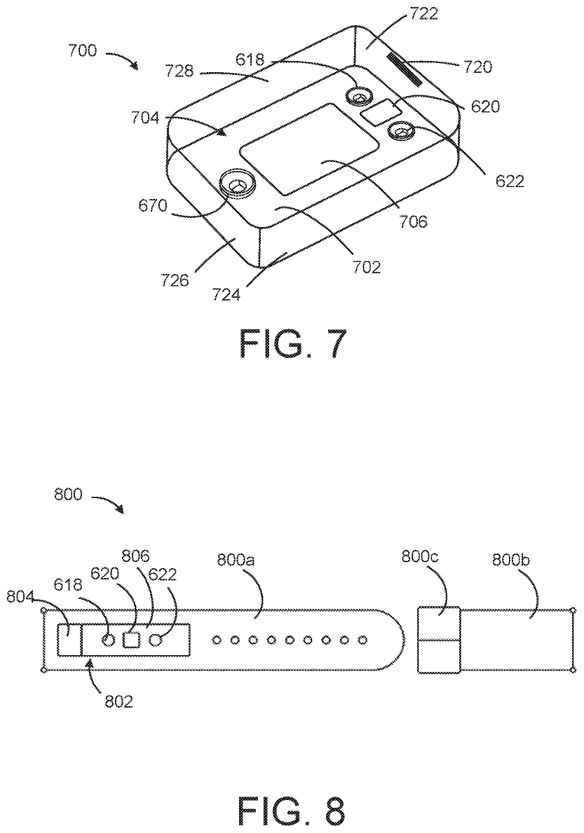

FIG. 7 illustrates a perspective view of an example implementation of an electronic auxiliary case 700 for obtaining biological data. The case 700 can function as a data collection system (and/or an accessory system) that uses sensors and processing components to measure and analyze physiological parameters of a person wearing the case 700 or wearing an electronic device coupled to the case 700.

The case 700 includes a stimulus source 702 with a number of sensors arranged within portions of the stimulus source 702. The stimulus source 702 in this example covers an entire base surface 704 of the case 700. The sensors shown here in example case 700 include the PPG sensor 618, the skin temperature sensor 620, the electrode 622, and the electrode 670. In this example, the sensors 618-622 are arranged in parallel to each other and within a perimeter associated with the stimulus source 702. The electrode 670 is also arranged within the surface perimeter of the stimulus source 702. In particular, the stimulus source 702 can at least partially surround the PPG sensor 618, the skin temperature sensor 620, the electrode 622, and/or the electrode 670.

Although not depicted, any number of sensors can be included in case 700, including, but not limited to an electroencephalogram (EEG) sensor, an inertial measurement unit (IMU) sensor, a heart rate (HR) sensor, an electrodermal activity (EDA) sensor, an electrocardiogram (ECG) sensor, and an electromyography (EMG) sensor, mechanical sensors, a pressure sensor, a motion sensor, a blood volume sensor, a muscle activity sensor, a sweat sensor, a tissue impedance sensor, or an environmental temperature sensor. Other arrangements of the stimulus source 702, the PPG sensor 618, the skin temperature sensor 620, the electrode 622, and the electrode 670 are of course possible.

The case 700 also includes an opening 706. The opening 706 can allow for viewing and access of a wearable electronic device (e.g., device 530) that is coupled, fastened, or otherwise encased or partially encased within case 700. The stimulus source 702 surrounds the opening 706.

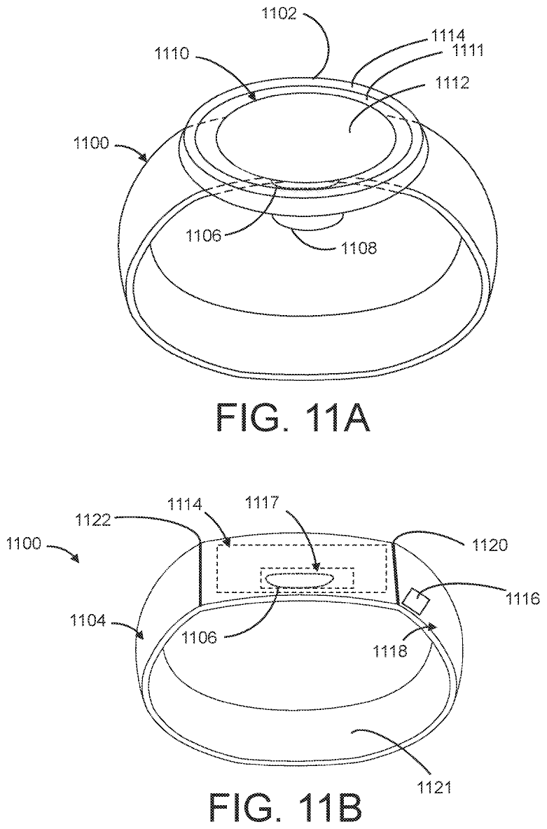

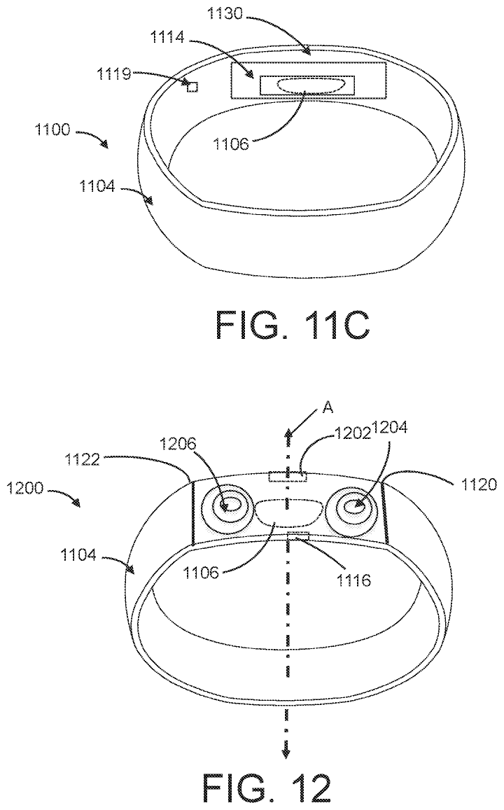

In some implementations, the case 700 can include one or more components of FIGS. 1B, 1C, and 9 and/or additional wiring (not shown) and/or circuitry (not shown) to couple the sensors 618-622, the electrode 670, or the stimulus source 702, electrically and communicatively to an electronic device (e.g., wearable electronic device 530) that is fully or partially encased within case 700. In some implementations, any one or more monitoring devices, accessory devices, straps, and/or cases shown in FIGS. 4A-8 and/or FIGS. 11A-12 can represent a wearable receptacle structure that can receive a physiological monitoring device. For example, the case 700 can represent a wearable receptacle structure that can receive device 530 within an opening of case 700. The physiological monitoring device (e.g., device 530) can be removably detachable from the wearable receptacle structure (e.g., case 700). For example, the device 530 can be removed from a wearable receptacle structure, such as case 700, to enable use of the features of device 530 without being coupled to (or encased within) the case 700.

In some implementations, the case 700 includes a receptacle portion (e.g., case 700) that defines a perimeter around the opening 706. The opening 706 can be sized or stretchable to receive the physiological monitoring device (e.g., device 530). In some implementations, the perimeter around the opening 706 also includes a first heat stimulus source 702. The case 700 can also receive or include a strap or band (e.g., band 408 or band 800) that can function to secure the receptacle portion (e.g., case 700) to a body of a user.

In some implementations, the band can have active components for obtaining measurements of physiological parameters associated with the user. The active components can include, but are not limited to sensors, stimulus sources, as described herein throughout this disclosure. For example, the strap can include a second heat stimulus source (e.g., within the band itself as described in FIGS. 11B-12). The second heat stimulus source can be in addition to the stimulus source 702 associated with the receptacle portion (e.g., case 700). In some implementations, the strap cannot include active components and can function to secure other components to a body portion of the user.

In some implementations, a portion of the perimeter of the receptacle portion (e.g., case 700) can further include a power source 720 to provide power to the first heat stimulus source 702 and/or any other sensors associated with the receptacle portion or the strap. An example perimeter of the receptacle portion (e.g., case 700) can include one or more walls 722, 724, 726, 728. Any of the one or more walls 722, 724, 726, and/or 728 can at least partially define a volume for housing electronics (e.g., PPG sensor 618, skin temperature sensor 620, electrodes, etc.) and the power source 720.

In some implementations, the receptacle portion (e.g., case 700) and the strap (e.g., band 408 or band 800) may form a unitary body, as shown in FIGS. 11A-12 below. The unitary body can integrate the receptacle portion and the strap portion into a single body. The unitary body can be formed of stretchable polymer material such that the strap can be stretched into position on a wrist of the user, for example. Similarly, the stretchable polymer material can allow for insertion of the physiological device into the wearable receptacle structure. In some implementations, the strap can be formed of non-stretchable materials and such a strap can include two or more portions that can be secured or fastened together to form an enclosure that can enable the strap to be worn on a body portion of the user.

FIG. 8 illustrates a top down view of yet another example implementation of an electronic band 800 for obtaining biological data and retaining a wearable electronic device. The electronic band 800 can be electrically and communicatively coupled to an electronic device (e.g., wearable electronic device 530) that is fully or partially coupled to band 800. The electronic device can be a wearable electronic device or a non-wearable electronic device that includes one or more of a sensor patch, a watch, a smart watch, a fitness tracker, a phone, a wearable battery, a camera, a bracelet, a ring, a controller, and smart glasses.

The electronic band 800 includes a band portion 800a, a band portion 800b, and a buckle fastener 800c to fasten band 800 around a body portion of a person. The band 800 can be removably coupled to a wearable electronic device such as device 530, device 102, or another electronic device connectable by one or more coupling portions, similar to coupling parts 602, 606 and/or one or more connectors 602a, 602b, 606a, 606b, as shown in FIG. 6A.

The band portion 800a includes a sensor system 802 and an electronics module 804 that can function together to measure and analyze physiological parameters of a person wearing the band 800. The sensor system 802 and electronics module 804 can be flexible within and around a surface of the band 800. The sensor system 802 shown in FIG. 8 can be arranged on band 800 to be placed in contact with a lateral side of a wrist of a person wearing the band 800. However, other surface locations of the band 800 can also receive the sensor system 802 and/or electronics module 804 are possible. In some implementations, the sensors 618, 620, 622 can also be in one or more openings/apertures of the band 800 to ensure partial, full, or near contact to a body site of the person wearing the band 800.

When the band 800 is coupled to another electronic device, as described above, the electronic band 800 can utilize and analyze data captured by sensors of the other electronic device. In addition, when the band 800 is coupled in such a way, the band 800 can assume control of a portion of the other electronic device to operate sensors in conjunction with the sensor system 802 of band 800, as described in detail herein.

The sensor system 802 can be powered and controlled by the electronics module 804. The band 800 includes a stimulus source 806 with a number of sensors arranged within portions of the stimulus source 806. The stimulus source 806 in this example is positioned on a portion of the band surface (shown at band portion 800a). The sensors shown here in example band 800 include the PPG sensor 618, the skin temperature sensor 620, and the electrode 622. In this example, the sensors 618-622 are arranged in parallel to each other and within a perimeter associated with the stimulus source 806.

Although not depicted, any number of sensors can be included in case 700, including, but not limited to an electroencephalogram (EEG) sensor, an inertial measurement unit (IMU) sensor, a heart rate (HR) sensor, an electrodermal activity (EDA) sensor, an electrocardiogram (ECG) sensor, and an electromyography (EMG) sensor, mechanical sensors, a pressure sensor, a motion sensor, a blood volume sensor, a muscle activity sensor, a sweat sensor, a tissue impedance sensor, or an environmental temperature sensor. Other arrangements of the stimulus source 806, the PPG sensor 618, the skin temperature sensor 620, and the electrode 622 are of course possible.

In some implementations, the electronics module 804 can include at least one processor (e.g., processor 922) and a transceiver (e.g., transmitter/receiver 920) and accompanying antennas. The at least one processor 922 can be communicatively coupled to one or more sensors 618-622, etc. and the stimulus source 806. The transceiver 920 can be communicatively connectable to the at least one processor 922, the stimulus source 806, the at least one sensor 618, 620, 622, etc., and the wearable electronic device (e.g., device 530). The processor 922 can be programmed to carry out program instructions for any or all of the methods described herein. For example, the processor 922 can execute computer-readable instructions that include monitoring a body site of a person having at least partial contact with at least one sensor 618, 620, 622, etc. of band 800. The processor 922 can further obtain, based on the monitoring, a physiological signal associated with the body site. The processor 922 can further generate, based on the physiological signal, a vasodilation estimate, a temperature estimate, a respiration rate, a pulse rate, a heart rate, a heart rate variability, a stress indication, a motion of the patient, core and/or skin surface temperatures, and/or hyperhidrosis for the body site. For example, the body site can be positioned in contact with the stimulus source 806 of the band 800 or a coupled wearable electronic device (e.g., device 530, device 102, or another electronic device). In some implementations, obtaining the physiological signal can occur after completion of at least one stimulus cycle generated by the stimulus source 806.

In some implementations, the band 800 also includes one or more input devices or output devices (e.g., I/O 918). The input devices can include software or hardware controls to control any number of functions of the band 800 and/or one or more functions of an electronic device (e.g., device 530) that is coupled to the band 800. The output devices can function to receive instructions and data from devices coupled to the band 800.

FIG. 9 is an example physiological monitoring system for obtaining and processing biological data. The monitoring system 900 can include an auxiliary device 902 (e.g., case 500, band 600, band 650, case 700, band 800, etc.) coupled to one or more wearable electronic devices 904 (e.g., device 530, device 102, or another electronic device). The auxiliary device 902 can be electrically and communicably coupled to wearable electronic device 904 and/or computing device 107 to selectively share sensor data, communication data, and/or control data amongst devices 902, 904, 107.

The auxiliary device 902 includes an accessory interface 906 in communication with a stimulus source 908, sensors 910, and electronics module 912. In some implementations, the electronics module 912 can include or incorporate the accessory interface 906.

The accessory interface 906 includes a communication means 914 and detection circuitry 916. The communication means 914 can represent wired or wireless architecture for communicating between accessory interface 906 and any or all of wearable electronic device 904, stimulus source 908, sensors 910, and/or electronics module 912. For example, the communication means 914 can include one or more communication lines for receiving data, signals, and/or control commands. The communication lines can couple the accessory interface 906 to any or all of wearable electronic device 904, stimulus source 908, sensors 910, and/or electronics module 912.

In some implementations, the auxiliary device 902 (e.g., the case 500, the band 600, the band 650, the case 700, or the band 800) can include or represent the accessory interface 906 for a wearable electronic device (e.g., wearable electronic device 530, 102, etc.). In such an example, the electronic accessory /uxiliary device 902 can include a first sensor system and the wearable electronic device 904 can include a second sensor system (e.g., including one or more sensors 104 of device 102).

The communication means 914 can include any number of communication signal traces/lines that form an electrical circuit between the wearable electronic device and an electronic accessory/auxiliary device 902. In some implementations, the communication means 914 is a wireless communication means that includes an electrical circuit for a transponder and/or receiver (or transceiver 920) to send and receive data wirelessly between the wearable electronic device and the auxiliary device 902.

The communication means 914 can also, or additionally, include one or more antennas and/or electrical circuits for a transponder and/or transceiver (e.g., optional transceiver 920) for sending and/or receiving data, signals, and/or control commands between the accessory interface 906 and any or all of wearable electronic device 904, stimulus source 908, sensors 910, and/or electronics module 912.

The detection circuitry 916 includes at least one processor 922, memory 924, and a detector 926. The detector 926 can represent a data detector to detect when data is sent or received from auxiliary device 902 and/or when data is sent or received from wearable electronic device 904 in implementations in which auxiliary device 902 is coupled to wearable electronic device 904. In some implementations, the detector 926 can represent a connection detector to detect when components are ready for use/access in wearable electronic device 904. In some implementations, the detector 926 can represent a sensor detector to detect which sensors are available for use/access in wearable electronic device 904. In some implementations, the detector 926 represents logic programmed to detect and select particular sensor capabilities of wearable electronic device 904 and for use with sensors 910 of auxiliary device 902. To select particular sensors/functions, a multiplexer circuitry 928 can be included in detector 926.

The stimulus source 908 can function in the same manner as stimulus source 410 on device 102 to emit heat or coolness. However, stimulus source 908 is instead installed in the auxiliary device 902 rather than the wearable electronic device 904. The stimulus source 908 can function with one or more sensors on the wearable electronic device 904 when the wearable electronic device 904 is communicatively coupled to auxiliary device 902, for example.

The sensors 910 can include any number of sensors including, but not limited to an electroencephalogram (EEG) sensor, an inertial measurement unit (IMU) sensor, a heart rate (HR) sensor, an electrodermal activity (EDA) sensor, an electrocardiogram (ECG) sensor, and an electromyography (EMG) sensor, mechanical sensors, a pressure sensor, a motion sensor, a blood volume sensor, a muscle activity sensor, a sweat sensor, a tissue impedance sensor, or an environmental temperature sensor.

The electronics module 912 can include optional power 930 to provide a power source to the auxiliary device 902 and devices within auxiliary device 902. The electronics module 912 can optionally include transceiver 920 functionality to communicate with sensors, memory, and/or processors of wearable electronic device 904.

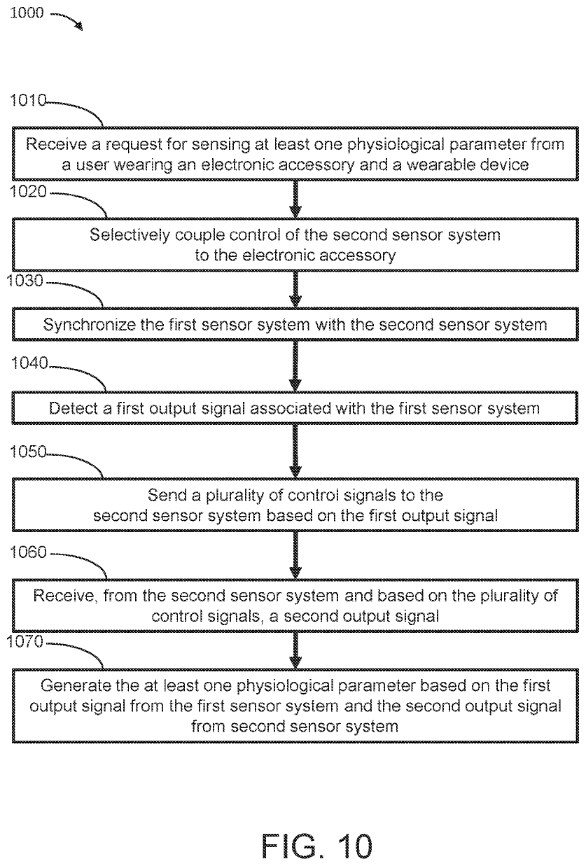

FIG. 10 illustrates a flow diagram of an example process 1000 for characterizing or monitoring a physiological response of a person using an auxiliary device. Such responses can be used by process 1000 (and the devices described herein) to generate at least one physiological parameter for the person wearing one or more of the wearable electronic devices described herein (e.g., device 102, device, 530, device 904) and when such devices are electrically and communicatively coupled to one of the accessory devices described herein (e.g., case 500, band 600, band 650, case 700, and/or band 800).

In some implementations, the process 1000 can be performed by an auxiliary device (e.g., devices 500, 600, 650, 700, 800) that is coupled to a wearable electronic device (e.g., devices 102, 530, 904, etc.). For example, the auxiliary device 902 can be coupled to the wearable electronic device 904. The coupled devices can be positioned on a body portion of a person to monitor a plurality of skin surface sites of the person. The auxiliary device 902 can include communication circuitry for transmitting and receiving data between the auxiliary device 902 (e.g., an electronic accessory) and the wearable electronic device 904. The auxiliary device 902 can include a first sensor system (e.g., sensors 910, accessory interface 906, stimulus source 908, and electronics module 912). The wearable electronic device 904 can include second sensor system (e.g., a heart rate monitor).

The auxiliary device 902 can also include detection circuitry to selectively couple the first sensor system to the second sensor system. The detection circuitry can be configured to selectively couple (e.g., electrically and/or communicatively) the first sensor system to the second sensor system to generate at least one physiological parameter for a person wearing the auxiliary device 902 and the wearable electronic device 904. In some implementations, the first sensor system and the second sensor system can be temporarily coupled until completion of a physiological measurement. In some implementations, the first sensor system and the second sensor system can be temporarily coupled until exhaustion of a predefined time period.

For example, at block 1010, the processor 922 of auxiliary device 902 can receive a request for sensing at least one physiological parameter of a user wearing the auxiliary device 902 (e.g., an electronic accessory) and the wearable electronic device 904. In some implementations, the auxiliary device 902 can be physically and/or electrically coupled to the wearable electronic device 904. In some implementations, the physical and/or electrical coupling does not allow sharing of data until the auxiliary device 902 and wearable electronic device 904 are coupled. The coupling can allow for auxiliary device 902 to poll wearable electronic device 904 to determine whether the second sensor system includes at least one sensor operable to generate additional measurements corresponding to the requested physiological parameter(s).

In response to determining that the second sensor system includes at least one sensor operable to generate additional measurements corresponding to the at least one physiological parameter, the process 1000 can include, at block 1020, selectively coupling control of the second sensor system to auxiliary device 902. The selectively coupling can include having the multiplexer 928 selecting at least one sensor of the first sensor system to synchronize to at least one sensor of the second sensor system. The accessory interface 906 of auxiliary device 902 and in particular, processor 922 can send instructions to the wearable electronic device 904 to synchronize the first sensor system with the second sensor system, at block 1030.

At block 1040, the process 1000 can include detecting a first output signal associated with the first sensor system of auxiliary device 902. For example, the processor 922 can detect a first output signal being generated by sensors 910 and/or stimulus source 908. The output can be a sensor output or a thermal stimulus output. Based on the detected first output signal, the process 1000 can include sending a plurality of control signals to the second sensor system, at block 1050. For example, if the detected first output signal is a signal indicating operation of the stimulus source 908, the plurality of control signals can be generated and sent to the wearable electronic device 904 to activate particular sensors in the second sensor system of wearable electronic device 904. Such activation can allow auxiliary device 902 to obtain measurements (e.g., output) from the particular sensors on wearable electronic device 904 at a predetermined time or time interval and responsive to the thermal heating or cooling of the user's body site. The measurements can represent a second output signal that is received at auxiliary device 902 and from the second sensor system of the wearable electronic device 904. The second output can be based on the plurality of control signals, at block 1060.

At block 1070, the process 1000 can generate the at least one physiological parameter for the person. For example, the processor 922 can assess the first output and the second output using one or more algorithms to analyze particular physiological data and generating the requested at least one physiological parameter based on the first output signal from the first sensor system (e.g., of auxiliary device 902) and the second output signal from second sensor system (of wearable electronic device 904).

FIG. 11A illustrates a perspective view of an example of an accessory device 1100 coupled to a wearable physiological monitoring device 1102. The accessory device 1100 can obtain biological data and/or can obtain biological data in combination with a wearable electronic device, such as the physiological monitoring device 1102. For example, device 1100 can work in combination with device 1102 when the devices are coupled together. In particular, the accessory device 1100 can be removably attached to the physiological monitoring device 1102.

The accessory device 1100 can include one or more sensors, stimulus sources, batteries, processors, antennas, or the like, and can function and communicate with the physiological monitoring device 1102 to obtain physiological measurements from a user wearing the combined devices 1100, 1102. The physiological monitoring device 1102 can include one or more of a sensor patch, a watch, a smart watch, a fitness tracker, a phone, a wearable battery, a camera, a bracelet, a ring, a controller, and smart glasses.

In some implementations, the accessory device 1100 can be a strap 1104 for removably attaching the physiological monitoring device 1102 to a user. For example, the accessory device 1100 can be integrated into the strap 1104. The physiological monitoring device 1102 can be inserted or otherwise fastened to the accessory device 1100 via the strap 1104 (or other securing means). The combined devices 1100, 1102 can be secured to a wrist of a user. In such examples, at least some components of the accessory device 1100 are integrated into the strap 1104, as described herein.

In some implementations, the strap 1104 is a flexible strap with one or more openings in which to receive a portion of the physiological monitoring device 1102. For example, the accessory device 1100 can be a strap with an opening 1106 (shown in FIG. 11B) to receive a portion of a bottom plate and/or one or more sidewalls of the physiological monitoring device 1102. In general, the accessory device 1100 can include a housing defining one or more openings in which to receive a sensor of the physiological monitoring device 1102. For example, the accessory device 1100 can include a first opening 1106 (FIG. 11B) to receive an optical sensor 1108. The optical sensor 1108 can be included as part of the physiological monitoring device 1102. When the accessory device 1100 is coupled to the physiological monitoring device 1102, the opening 1106 can be substantially aligned with sensor 1108 to secure the strap 1104 of the accessory device 1100 to the physiological monitoring device 1102.

The opening 1106 can enable the optical sensor 1108 (or other sensor) of the wearable physiological monitoring device 1102 to be placed in contact with a body site of a user wearing the physiological monitoring device 1102 coupled to the accessory device 1100. The optical sensor 1108 can be a PPG sensor that can be placed in contact with a skin surface associated with the body site of the user when the accessory device 1100 is coupled to the physiological monitoring device 1102 and worn by the user. In some implementations, the housing of the accessory device 1100 can include a second opening 1110. The opening 1110 can at least partially surround a portion of the device 1102 such that a display 1112 of the physiological monitoring device 1102 is viewable through the opening 1110 of the accessory device 1100.

In some implementations, the optical sensor 1108 is not included on the accessory device 1100, but is instead part of the physiological monitoring device 1102. The devices 1100, 1102 can function together to operate the optical sensor and one or more heat stimulus sources (e.g., heat stimulus source 1114 shown in FIGS. 11B, 11C, 12) to obtain PPG sensor readings, as described in detail herein.

FIG. 11B illustrates a perspective view of the accessory device of FIG. 11A. In this example, the accessory device 1100 further includes a heat stimulus source 1114 and a battery 1116 configured to power the heat stimulus source 1114. For example, the battery 1116 can provide power to the strap 1104 to energize the heat stimulus source 1114. In some implementations, the battery 1116 (or other power source) can further provide power to other onboard components including, but not limited to processors, antennas, and other onboard sensors. In some implementations, the battery 1116 is positioned on an outer surface of the strap 1104 to avoid contact with a skin of the user. In some implementations, the battery 1116 is positioned on or in at least one sidewall of a housing of the accessory device 1100 or the accessory device 1200.

In some implementations, the accessory device 1102 can include one or both of a first connector 1120 and a second connector 1122 that can fasten the accessory device 1100 to the physiological monitoring device 1102. In some implementations, the accessory device can include a number of openings that are couplable to portions of the physiological monitoring device 1102, which can provide a tension fit to retain device 1102 within device 1100.

The accessory device 1100 can be electrically and communicatively coupled to the physiological monitoring device 1102 when the two devices 1100, 1102 are fully or partially coupled. In such examples, the battery 1116 and/or one or more heat stimulus source (e.g., heat stimulus source 1114) can be shared between the two devices 1100, 1102.

In some implementations, the heat stimulus source 1114 can be positioned outside of an outer periphery (e.g., perimeter 1111 shown in FIG. 11A) of the wearable physiological monitoring device 1102. For example, the heat stimulus source 1114 can be integrated into the strap 1104 beyond the external perimeter 1111 of device 1102. Although the heat stimulus source 1114 can be located further from the components of the physiological monitoring device 1102, additional components and heat emitters can be placed within the perimeter 1111 of device 1102, but can be wired to battery 1116 and heat stimulus source 1114 to receive heat and signals.

Referring again to FIG. 11B, the heat stimulus source 1114 can be included on the accessory device 1100 to assist in obtaining vasodilation measurements or other measurements (e.g., hydration, edema, EDA, etc.) of the user wearing the combined devices 1100, 1102. For example, the optical sensor 1108 (FIG. 11A) can be received within opening 1106, and when seated within opening 1106, the optical sensor 1108 can be enclosed within an area 1117 of the heat stimulus source 1114. In some implementations, the optical sensor 1108 can be positioned on the strap 1104 to measure a vasodilation response on a ventral side of the wrist, for example, when the combined devices 1100, 1102 are worn on the wrist of the user.

In some implementations, the heat stimulus source 1114 can be placed adjacent to the sensor 1108. For example, the heat stimulus source 1114 can be installed within a portion of the strap of the accessory device 1100. For example, the heat stimulus source 1114 can be integrated within (e.g., between) an outer surface 1118 and an inner surface 1121 of the accessory device 1100, thereby hidden within the strap 1104 of device 1100. In some implementations, the heat stimulus source 1114 can substantially cover an inner surface 1121 of the strap 1104 that is in contact with a skin of the user.

FIG. 11C illustrates another perspective view of the accessory device 1100 of FIG. 11A. In this example, a surface 1130 of the accessory device 1100 can be placed in contact with a wrist of a user to monitor and record physiological parameters of the user. As shown, the physiological monitoring device 1102 is not coupled to the accessory device 1100, although the opening 1106 can receive sensor 1108 of device 1102 and heat stimulus source 1114 can cause warming of a body site of the user when the device 1100 is worn by the user.

In this example, the accessory device 1100 further includes one or more temperature sensors 1119 (e.g., skin temperature sensor 620) that can function with the heat stimulus source 1114 to obtain vasodilation measurements, as described in detail herein. Although the temperature sensor 1119 is depicted within a particular distance from the heat stimulus source 1114, the temperature sensor 1119 can instead be placed in any location along the band (e.g., strap 1104) of the accessory device 1100.

FIG. 12 illustrates a perspective view of another example accessory device 1200. The accessory device 1200 includes the opening 1106 for a sensor 1108 (shown in FIG. 11A) and a battery 1116 to power one or more of a heat stimulus source 1204 and/or a heat stimulus source 1206. Although accessory device 1200 includes two heat stimulus sources 1204, 1206, any number of heat stimulus sources can be provided on or within portions of accessory device 1200. For example, any or all of a strap 1104 can be lined with heat stimulus sources to warm a portion of a body site of the user. For example, the heat stimulus source 1204 and/or heat stimulus source 1206 can be positioned along at least some portions of a periphery of the opening 1106.

In some implementations, the heat stimulus sources 1204, 1206 include concentric heating elements. For example, the heat stimulus sources 1204, 1206 are depicted in FIG. 12 as substantially concentric with respect to axis A. That is, the heat stimulus sources 1204, 1206 can be oriented such that in a cross section of the accessory device 1200 taken perpendicular to the axis A, the heating stimulus sources 1204, 1206 would be seen as concentric circles. The concentric heating elements (e.g., heat stimulus sources 1204, 1206) can include metal or ceramic filaments or wires that can be substantially helically wound that can emit heat when energized with power.

Although heat stimulus sources 1204, 1206 are depicted as concentric heating elements, one of skill in the art will appreciate that any heating element or any shape of heating element can be contemplated. In addition, although two concentric heating elements (e.g., heat stimulus sources 1204, 1206) are depicted in the embodiment of FIG. 12, one of skill in the art will appreciate that a single concentric heating element can replace heat stimulus sources 1204, 1206 in the embodiment of FIG. 12 and/or any embodiment described herein that includes a heat stimulus source.

In some implementations, the heat stimulus sources 1204, 1206 can be included on the strap 1104 to cause warming of a body site of the user wearing device 1200. For example, a first heat stimulus source (e.g., stimulus source 1204) can be positioned adjacent to the first connector 1120 while a second heat stimulus source (e.g., stimulus source 1206) can be positioned adjacent to the second connector 1122. The first and second heat stimulus sources 1204, 1206 can be operated to warm the body site around a perimeter of the sensor 1108, for example, when the accessory device 1200 is coupled to the physiological monitoring device 1102. The warming of the body site can, for example, enable accurate PPG sensing to occur via the sensor 1108 on the physiological monitoring device 1202.

The accessory devices described herein can also include one or more hardware processors (e.g., processors 922) which can be configured to control the heat stimulus sources 1114, 1204, 1206, etc. For example, the one or more hardware processors 922 can be programmed with logic and/or instructions for controlling and operating the heat stimulus sources 1114, 1204, 1206, etc. The logic and/or instructions can be stored in a memory as described in detail herein. In addition, the one or more hardware processors can perform processes as discussed herein for detection of input conditions and control of output conditions.

The accessory devices described herein can further include one or more antennas within a communication means (e.g., communication means 914). For example, the one or more antennas can communicate via a transceiver (e.g., transmitter/receiver 920). The one or more antennas can enable communication between one or more accessory device 1100, 1200, etc. and the wearable physiological monitoring device 1102. The one or more antennas can also enable communication with a computing device or other device external to the accessory device 1100 or accessory device 1200, for example.

Although not depicted, any number of sensors can be included in accessory devices 1100, 1200, including, but not limited to additional PPG sensors, an electroencephalogram (EEG) sensor, an inertial measurement unit (IMU) sensor, a heart rate (HR) sensor, an electrodermal activity (EDA) sensor, an electrocardiogram (ECG) sensor, and an electromyography (EMG) sensor, mechanical sensors, a pressure sensor, a motion sensor, a blood volume sensor, a muscle activity sensor, a sweat sensor, a tissue impedance sensor, or a body or environmental temperature sensor. Other arrangements of the heat stimulus source(s) 1114, the PPG sensor 1108, and the temperature sensor 1119 are of course possible.

The systems and methods of the implementations described herein and/or variations thereof can be embodied and/or implemented at least in part as a machine configured to receive a computer-readable medium storing computer-readable instruction. The instructions are executed by computer-executable components preferably integrated with the system and one or more portions of the hardware processor on the device for detecting stroke and/or computing device. The computer-readable medium can be stored on any suitable computer-readable media (e.g., memory 118) such as RAMs, ROMs, flash memory, EEPROMs, optical devices (e.g., CD or DVD), hard drives, floppy drives, or any suitable device. The computer-executable component can be a general or application-specific hardware processor, but any suitable dedicated hardware or hardware/firmware combination can alternatively or additionally execute the instructions.

As used in the description and claims, the singular form “a”, “an” and “the” include both singular and plural references unless the context clearly dictates otherwise. For example, the term “signal” may include, and is contemplated to include, a plurality of signals. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5 percent, 1 percent or 0.1 percent. All numerical ranges provided herein are inclusive of the stated start and end numbers. The term “substantially” indicates mostly (i.e., greater than 50%) or essentially all of a device, substance, or composition.

The term “horizontal” as used herein is defined as a plane parallel to the conventional plane or surface of a heating element (e.g., heat source 410), regardless of its orientation. The term “vertical” refers to a direction perpendicular to the horizontal as just defined. Terms, such as “on”, “above”, “below”, “bottom”, “top”, “side” (as in “sidewall”), “higher”, “lower”, “over”, and “under”, are defined with respect to the horizontal plane.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Implementations defined by each of these transitional terms are within the scope of this disclosure.

The examples and illustrations included herein show, by way of illustration and not of limitation, specific implementations in which the subject matter may be practiced. Other implementations may be utilized and derived therefrom, such that structural and logical substitutions and changes may be made without departing from the scope of this disclosure. Such implementations of the inventive subject matter may be referred to herein individually or collectively by the term “invention” merely for convenience and without intending to voluntarily limit the scope of this application to any single invention or inventive concept, if more than one is in fact disclosed. Thus, although specific implementations have been illustrated and described herein, any arrangement calculated to achieve the same purpose may be substituted for the specific implementations shown. This disclosure is intended to cover any and all adaptations or variations of various implementations. Combinations of the above implementations, and other implementations not specifically described herein, will be apparent to those of skill in the art upon reviewing the above description.

1. An accessory device configured to be removably attached to a wearable physiological monitoring device, said wearable physiological monitoring device including at least an optical sensor configured to be positioned in proximity to a wrist of a user, the accessory device comprising:
a heat stimulus source;
a battery configured to power the heat stimulus source; and
a housing, said housing comprising a first opening, said first opening configured to enable the optical sensor of the wearable physiological monitoring device to contact a skin of a user wearing the wearable physiological monitoring device.

2. (canceled)

3. The accessory device of claim 1, wherein the housing further comprises a second opening, said second opening being configured to enable a display of the wearable physiological monitoring device to be viewable through the second opening.

4. The accessory device of claim 1, wherein the heat stimulus source is positioned along at least some portions of a periphery of the first opening.

5. The accessory device of claim 4, wherein the heat stimulus source comprises concentric heating elements.

6. The accessory device of claim 1, wherein the housing comprises side walls and wherein the battery is positioned along at least one of the side walls.

7. The accessory device of claim 1, further comprising a hardware processor configured to control the heat stimulus source.

8. The accessory device of claim 1, further comprising one or more antennas configured to communicate with the wearable physiological monitoring device.

9. The accessory device of claim 1, further comprising one or more antennas configured to communicate with a computing device.

10. The accessory device of claim 1, wherein the wearable physiological monitoring device does not include any optical sensor.

11. The accessory device of claim 1, further comprising one or more temperature sensors.

12. The accessory device of claim 1, wherein the wearable physiological monitoring device is configured to be secured to a wrist of a user.

13. The accessory device of claim 12, further comprising a strap configured to secure the wearable physiological monitoring device to the user, wherein at least some components of the accessory device are integrated into the strap.

14. The accessory device of claim 1, wherein the heat stimulus source is positioned outside of an outer periphery of the wearable physiological monitoring device.

15. The accessory device of claim 1, further comprising a strap configured to removably attach to the wearable physiological monitoring device, wherein the accessory device is integrated into the strap.
